SEQUENCE LISTING (22 sequences)

SEQ ID NO: 1 - DNA, 41 bp, Artificial Sequence
  Chemically synthesized primer gal2HC_5′
actagtaggaggtaacttatgaaactgaaacgtttgatggc

SEQ ID NO: 2 - DNA, 39 bp, Artificial Sequence
  Chemically synthesized primer XbaI_VhCH1_R
tctagatcattactaaacgcgcttgtcacctttcgtgtt

SEQ ID NO: 3 - DNA, 39 bp, Artificial Sequence
  Chemically synthesized primer XbaI_pbp_F
tctagaaggaggtaacttatgaaactgaaacgtttgatg

SEQ ID NO: 4 - DNA, 39 bp, Artificial Sequence
  Chemically synthesized primer XhoI_L_R
ctcgagctatcattagcactcgccgcgattaaacgactt

SEQ ID NO: 5 - DNA, 46 bp, Artificial Sequence
  Chemically synthesized primer gal2HC_5′
actagtaggaggtaacttatgaaactgaaacgtttgatggcggcaa

SEQ ID NO: 6 - DNA, 35 bp, Artificial Sequence
  Chemically synthesized primer CH1_rev
cgtctagattatcactagcacgatttcggctcaac

SEQ ID NO: 7 - DNA, 36 bp, Artificial Sequence
  Chemically synthesized primer CH1_hinge
gctctagattactatcagcacggcgggcaggtatgc

SEQ ID NO: 8 - protein, 97 aa, Artificial Sequence
  Chemically synthesized plasmid gal2 FAB HC-JM(146)
STKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 9 - protein, 102 aa, Artificial Sequence
  Chemically synthesized plasmid gal2 Fab1 Hc YS (122)
STKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 10 - protein, 111 aa, Artificial Sequence
  Chemically synthesized plasmid gal2 Fab2 YS (122)
STKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPC

SEQ ID NO: 11 - protein, 102 aa, Artificial Sequence; identical to SEQ ID NO: 9
  Chemically synthesized consensus sequence

SEQ ID NO: 12 - DNA, 12 bp, Artificial
  Chemically synthesized  pbp heavy chain
aggaggtaactt

SEQ ID NO: 13 - DNA, 12 bp, Artificial
  Chemically synthesized pbp light chain
aggaggtaactt

SEQ ID NO: 14 - DNA, 1620 bp, Artificial
  Chemically synthesized Coding Region of pDOW1196
tttattctgaaatgagctgttgacaattaatcatcggctcgtataatgtgtggaattgtgagcggataacaatttcacacaggaaacagaattttaatctactagtaggaggtaacttatgaaactgaaacgtttgatggcggcaatgacttttgtcgctgctggcgttgcgaccgccaacgcggtggcccaggtgcagctgcaggagtcgggcccaggactggtgaagccttcggagaccctgtccctcacctgcactgtctctggtggttccatcagtagttatcactggagctggatccggcagcccccagggaagggactggagtggattgggtatatctattacagtgggagcaccaactacaacccctccctcaagaatcgagtcaccatatctgtagacacgtccaagaaccagttctccctgaacctgaggtctgtgaccgctgcagacacggccgtgtattactgtgcgcgaggaacgtatggcccagccggagatgcttttgatatctgggggcaagggaccacggtcaccgtctcgtcggcctccacgaaaggcccgagcgtgttcccgctggcgccaagctccaagagcaccagcggcggcaccgccgcgctgggttgtctcgtcaaagattacttccccgaaccggtgaccgtgtcgtggaactccggggcgctgaccagcggtgtccataccttccctgccgtgctccagtcctccggcctgtattccctgagctcggtggtgaccgtgccgtcgtcgagcttgggcacccaaacctacatctgcaacgtcaaccataagccctccaacacgaaagttgacaagcgcgtttagtaatgatctagaaggaggtaacttatgaaactgaaacgtttgatggcggcaatgacttttgtcgctgctggcgttgcgaccgccaacgcggtggccgacatccagatgacccagtctccttccaccctgtctgcatctattggagacagagtcaccatcacctgccgggccagtgagggtatttatcactggttggcctggtatcagcagaagccagggaaagcccctaaactcctgatctataaggcctctagtttagccagtggggccccatcaaggttcagcggcagtggatctgggacagatttcactctcaccatcagcagcctgcagcctgatgattttgcaacttattactgccaacaatatagtaattatccgctcactttcggcggagggaccaagctggagatcaaacgtgcggtcgccgccccgtcggttttcattttcccgccatcggatgagcagctcaagtcgggcacggcgagcgtggtctgcctgctcaacaacttttacccgcgcgaggccaaggtgcagtggaaggtcgacaacgccctgcagtcgggcaacagccaggagtcggtcaccgagcaggatagcaaggattccacctattccctcagctcgaccctgacgctgagcaaggccgattatgagaagcataaagtttacgcttgtgaagtgacccaccagggcctgagcagcccggtgaccaagtcgtttaatcgcggcgagtgctaatgatagctcgagcccaaaacgaaaggctcagt

SEQ ID NO: 15 - protein, 480 aa, Artificial
  Chemically synthesized Amino Acid sequence corresponding to Coding Region of pDOW1196
MKLKRLMAAMTFVAAGVATANAVAQVQLQESGPGLVKPSETLSLTCTVSGGSISSYHWSWIRQPPGKGLEWIGYIYYSGSTNYNPSLKNRVTISVDTSKNQFSLNLRSVTAADTAVYYCARGTYGPAGDAFDIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVMKLKRLMAAMTFVAAGVATANAVADIQMTQSPSTLSASIGDRVTITCRASEGIYHWLAWYQQKPGKAPKLLIYKASSLASGAPSRFSGSGSGTDFTLTISSLQPDDFATYYCQQYSNYPLTFGGGTKLEIKRAVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - DNA, 1620 bp, Artificial
  Chemically synthesized Complementary Strand of Coding Region of pDOW1196
aaataagactttactcgacaactgttaattagtagccgagcatattacacaccttaacactcgcctattgttaaagtgtgtcctttgtcttaaaattagatgatcatcctccattgaatactttgactttgcaaactaccgccgttactgaaaacagcgacgaccgcaacgctggcggttgcgccaccgggtccacgtcgacgtcctcagcccgggtcctgaccacttcggaagcctctgggacagggagtggacgtgacagagaccaccaaggtagtcatcaatagtgacctcgacctaggccgtcgggggtcccttccctgacctcacctaacccatatagataatgtcaccctcgtggttgatgttggggagggagttcttagctcagtggtatagacatctgtgcaggttcttggtcaagagggacttggactccagacactggcgacgtctgtgccggcacataatgacacgcgctccttgcataccgggtcggcctctacgaaaactatagacccccgttccctggtgccagtggcagagcagccggaggtgctttccgggctcgcacaagggcgaccgcggttcgaggttctcgtggtcgccgccgtggcggcgcgacccaacagagcagtttctaatgaaggggcttggccactggcacagcaccttgaggccccgcgactggtcgccacaggtatggaagggacggcacgaggtcaggaggccggacataagggactcgagccaccactggcacggcagcagctcgaacccgtgggtttggatgtagacgttgcagttggtattcgggaggttgtgctttcaactgttcgcgcaaatcattactagatcttcctccattgaatactttgactttgcaaactaccgccgttactgaaaacagcgacgaccgcaacgctggcggttgcgccaccggctgtaggtctactgggtcagaggaaggtgggacagacgtagataacctctgtctcagtggtagtggacggcccggtcactcccataaatagtgaccaaccggaccatagtcgtcttcggtccctttcggggatttgaggactagatattccggagatcaaatcggtcaccccggggtagttccaagtcgccgtcacctagaccctgtctaaagtgagagtggtagtcgtcggacgtcggactactaaaacgttgaataatgacggttgttatatcattaataggcgagtgaaagccgcctccctggttcgacctctagtttgcacgccagcggcggggcagccaaaagtaaaagggcggtagcctactcgtcgagttcagcccgtgccgctcgcaccagacggacgagttgttgaaaatgggcgcgctccggttccacgtcaccttccagctgttgcgggacgtcagcccgttgtcggtcctcagccagtggctcgtcctatcgttcctaaggtggataagggagtcgagctgggactgcgactcgttccggctaatactcttcgtatttcaaatgcgaacacttcactgggtggtcccggactcgtcgggccactggttcagcaaattagcgccgctcacgattactatcgagctcgggttttgctttccgagtca

SEQ ID NO: 17 - DNA, 1612 bp, Artificial
  Chemically synthesized Coding region of pDOW3716
ctgaaatgagctgttgacaattaatcatcggctcgtataatgtgtggaattgtgagcggataacaatttcacacaggaaacagaattttaatctactagtaggaggtaacttatgaaactgaaacgtttgatggcggcaatgacttttgtcgctgctggcgttgcgaccgccaacgcggtggcccaggtgcagctgcaggagtcgggcccaggactggtgaagccttcggagaccctgtccctcacctgcactgtctctggtggttccatcagtagttatcactggagctggatccggcagcccccagggaagggactggagtggattgggtatatctattacagtgggagcaccaactacaacccctccctcaagaatcgagtcaccatatctgtagacacgtccaagaaccagttctccctgaacctgaggtctgtgaccgctgcagacacggccgtgtattactgtgcgcgaggaacgtatggcccagccggagatgcttttgatatctgggggcaagggaccacggtcaccgtctcgtcggcctccacgaaaggcccgagcgtgttcccgctggcgccaagctccaagagcaccagcggcggcaccgccgcgctgggttgtctcgtcaaagattacttccccgaaccggtgaccgtgtcgtggaactccggggcgctgaccagcggtgtccataccttccctgccgtgctccagtcctccggcctgtattccctgagctcggtggtgaccgtgccgtcgtcgagcttgggcacccaaacctacatctgcaacgtcaaccataagccctccaacacgaaagttgacaagcgcgttgagccgaaatcgtgctagtgataatctagaaggaggtaacttatgaaactgaaacgtttgatggcggcaatgacttttgtcgctgctggcgttgcgaccgccaacgcggtggccgacatccagatgacccagtctccttccaccctgtctgcatctattggagacagagtcaccatcacctgccgggccagtgagggtatttatcactggttggcctggtatcagcagaagccagggaaagcccctaaactcctgatctataaggcctctagtttagccagtggggccccatcaaggttcagcggcagtggatctgggacagatttcactctcaccatcagcagcctgcagcctgatgattttgcaacttattactgccaacaatatagtaattatccgctcactttcggcggagggaccaagctggagatcaaacgtgcggtcgccgccccgtcggttttcattttcccgccatcggatgagcagctcaagtcgggcacggcgagcgtggtctgcctgctcaacaacttttacccgcgcgaggccaaggtgcagtggaaggtcgacaacgccctgcagtcgggcaacagccaggagtcggtcaccgagcaggatagcaaggattccacctattccctcagctcgaccctgacgctgagcaaggccgattatgagaagcataaagtttacgcttgtgaagtgacccaccagggcctgagcagcccggtgaccaagtcgtttaatcgcggcgagtgctaatgatagctcgagccc

SEQ ID NO: 18 - protein, 479 aa, Artificial
  Chemically synthesized Amino Acid Sequence Corresponding to Coding region of pDOW3716
MAAMTFVAAGVATANAVAQVQLQESGPGLVKPSETLSLTCTVSGGSISSYHWSWIRQPPGKGLEWIGYIYYSGSTNYNPSLKNRVTISVDTSKNQFSLNLRSVTAADTAVYYCARGTYGPAGDAFDIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCMKLKRLMAAMTFVAAGVATANAVADIQMTQSPSTLSASIGDRVTITCRASEGIYHWLAWYQQKPGKAPKLLIYKASSLASGAPSRFSGSGSGTDFTLTISSLQPDDFATYYCQQYSNYPLTFGGGTKLEIKRAVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - DNA, 1612 bp, Artificial
  Chemically synthesized Complementary Strand of Coding region of pDOW3716
gactttactcgacaactgttaattagtagccgagcatattacacaccttaacactcgcctattgttaaagtgtgtcctttgtcttaaaattagatgatcatcctccattgaatactttgactttgcaaactaccgccgttactgaaaacagcgacgaccgcaacgctggcggttgcgccaccgggtccacgtcgacgtcctcagcccgggtcctgaccacttcggaagcctctgggacagggagtggacgtgacagagaccaccaaggtagtcatcaatagtgacctcgacctaggccgtcgggggtcccttccctgacctcacctaacccatatagataatgtcaccctcgtggttgatgttggggagggagttcttagctcagtggtatagacatctgtgcaggttcttggtcaagagggacttggactccagacactggcgacgtctgtgccggcacataatgacacgcgctccttgcataccgggtcggcctctacgaaaactatagacccccgttccctggtgccagtggcagagcagccggaggtgctttccgggctcgcacaagggcgaccgcggttcgaggttctcgtggtcgccgccgtggcggcgcgacccaacagagcagtttctaatgaaggggcttggccactggcacagcaccttgaggccccgcgactggtcgccacaggtatggaagggacggcacgaggtcaggaggccggacataagggactcgagccaccactggcacggcagcagctcgaacccgtgggtttggatgtagacgttgcagttggtattcgggaggttgtgctttcaactgttcgcgcaactcggctttagcacgatcactattagatcttcctccattgaatactttgactttgcaaactaccgccgttactgaaaacagcgacgaccgcaacgctggcggttgcgccaccggctgtaggtctactgggtcagaggaaggtgggacagacgtagataacctctgtctcagtggtagtggacggcccggtcactcccataaatagtgaccaaccggaccatagtcgtcttcggtccctttcggggatttgaggactagatattccggagatcaaatcggtcaccccggggtagttccaagtcgccgtcacctagaccctgtctaaagtgagagtggtagtcgtcggacgtcggactactaaaacgttgaataatgacggttgttatatcattaataggcgagtgaaagccgcctccctggttcgacctctagtttgcacgccagcggcggggcagccaaaagtaaaagggcggtagcctactcgtcgagttcagcccgtgccgctcgcaccagacggacgagttgttgaaaatgggcgcgctccggttccacgtcaccttccagctgttgcgggacgtcagcccgttgtcggtcctcagccagtggctcgtcctatcgttcctaaggtggataagggagtcgagctgggactgcgactcgttccggctaatactcttcgtatttcaaatgcgaacacttcactgggtggtcccggactcgtcgggccactggttcagcaaattagcgccgctcacgattactatcgagctcggg

SEQ ID NO: 20 - DNA, 1639 bp, Artificial
  Chemically synthesized Coding region of pDOW3717
ctgaaatgagctgttgacaattaatcatcggctcgtataatgtgtggaattgtgagcggataacaatttcacacaggaaacagaattttaatctactagtaggaggtaacttatgaaactgaaacgtttgatggcggcaatgacttttgtcgctgctggcgttgcgaccgccaacgcggtggcccaggtgcagctgcaggagtcgggcccaggactggtgaagccttcggagaccctgtccctcacctgcactgtctctggtggttccatcagtagttatcactggagctggatccggcagcccccagggaagggactggagtggattgggtatatctattacagtgggagcaccaactacaacccctccctcaagaatcgagtcaccatatctgtagacacgtccaagaaccagttctccctgaacctgaggtctgtgaccgctgcagacacggccgtgtattactgtgcgcgaggaacgtatggcccagccggagatgcttttgatatctgggggcaagggaccacggtcaccgtctcgtcggcctccacgaaaggcccgagcgtgttcccgctggcgccaagctccaagagcaccagcggcggcaccgccgcgctgggttgtctcgtcaaagattacttccccgaaccggtgaccgtgtcgtggaactccggggcgctgaccagcggtgtccataccttccctgccgtgctccagtcctccggcctgtattccctgagctcggtggtgaccgtgccgtcgtcgagcttgggcacccaaacctacatctgcaacgtcaaccataagccctccaacacgaaagttgacaagcgcgttgagccgaaatcgtgcgacaagacgcatacctgcccgccgtgctgatagtaatctagaaggaggtaacttatgaaactgaaacgtttgatggcggcaatgacttttgtcgctgctggcgttgcgaccgccaacgcggtggccgacatccagatgacccagtctccttccaccctgtctgcatctattggagacagagtcaccatcacctgccgggccagtgagggtatttatcactggttggcctggtatcagcagaagccagggaaagcccctaaactcctgatctataaggcctctagtttagccagtggggccccatcaaggttcagcggcagtggatctgggacagatttcactctcaccatcagcagcctgcagcctgatgattttgcaacttattactgccaacaatatagtaattatccgctcactttcggcggagggaccaagctggagatcaaacgtgcggtcgccgccccgtcggttttcattttcccgccatcggatgagcagctcaagtcgggcacggcgagcgtggtctgcctgctcaacaacttttacccgcgcgaggccaaggtgcagtggaaggtcgacaacgccctgcagtcgggcaacagccaggagtcggtcaccgagcaggatagcaaggattccacctattccctcagctcgaccctgacgctgagcaaggccgattatgagaagcataaagtttacgcttgtgaagtgacccaccagggcctgagcagcccggtgaccaagtcgtttaatcgcggcgagtgctaatgatagctcgagccc

SEQ ID NO: 21 - protein, 488 aa, Artificial
  Chemically synthesized Amino Acid Sequence Corresponding to Coding region of pDOW3717
MAAMTFVAAGVATANAVAQVQLQESGPGLVKPSETLSLTCTVSGGSISSYHWSWIRQPPGKGLEWIGYIYYSGSTNYNPSLKNRVTISVDTSKNQFSLNLRSVTAADTAVYYCARGTYGPAGDAFDIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCMKLKRLMAAMTFVAAGVATANAVADIQMTQSPSTLSASIGDRVTITCRASEGIYHWLAWYQQKPGKAPKLLIYKASSLASGAPSRFSGSGSGTDFTLTISSLQPDDFATYYCQQYSNYPLTFGGGTKLEIKRAVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - DNA, 1639 bp, Artificial
  Chemically synthesized Complementary Strand of Coding region of pDOW3717
gactttactcgacaactgttaattagtagccgagcatattacacaccttaacactcgcctattgttaaagtgtgtcctttgtcttaaaattagatgatcatcctccattgaatactttgactttgcaaactaccgccgttactgaaaacagcgacgaccgcaacgctggcggttgcgccaccgggtccacgtcgacgtcctcagcccgggtcctgaccacttcggaagcctctgggacagggagtggacgtgacagagaccaccaaggtagtcatcaatagtgacctcgacctaggccgtcgggggtcccttccctgacctcacctaacccatatagataatgtcaccctcgtggttgatgttggggagggagttcttagctcagtggtatagacatctgtgcaggttcttggtcaagagggacttggactccagacactggcgacgtctgtgccggcacataatgacacgcgctccttgcataccgggtcggcctctacgaaaactatagacccccgttccctggtgccagtggcagagcagccggaggtgctttccgggctcgcacaagggcgaccgcggttcgaggttctcgtggtcgccgccgtggcggcgcgacccaacagagcagtttctaatgaaggggcttggccactggcacagcaccttgaggccccgcgactggtcgccacaggtatggaagggacggcacgaggtcaggaggccggacataagggactcgagccaccactggcacggcagcagctcgaacccgtgggtttggatgtagacgttgcagttggtattcgggaggttgtgctttcaactgttcgcgcaactcggctttagcacgctgttctgcgtatggacgggcggcacgactatcattagatcttcctccattgaatactttgactttgcaaactaccgccgttactgaaaacagcgacgaccgcaacgctggcggttgcgccaccggctgtaggtctactgggtcagaggaaggtgggacagacgtagataacctctgtctcagtggtagtggacggcccggtcactcccataaatagtgaccaaccggaccatagtcgtcttcggtccctttcggggatttgaggactagatattccggagatcaaatcggtcaccccggggtagttccaagtcgccgtcacctagaccctgtctaaagtgagagtggtagtcgtcggacgtcggactactaaaacgttgaataatgacggttgttatatcattaataggcgagtgaaagccgcctccctggttcgacctctagtttgcacgccagcggcggggcagccaaaagtaaaagggcggtagcctactcgtcgagttcagcccgtgccgctcgcaccagacggacgagttgttgaaaatgggcgcgctccggttccacgtcaccttccagctgttgcgggacgtcagcccgttgtcggtcctcagccagtggctcgtcctatcgttcctaaggtggataagggagtcgagctgggactgcgactcgttccggctaatactcttcgtatttcaaatgcgaacacttcactgggtggtcccggactcgtcgggccactggttcagcaaattagcgccgctcacgattactatcgagctcggg

(19) United States
(12) Patent Application Publication
(10) Pub. No.: US 2009/0042254 A1
(43) Pub. Date: Feb. 12, 2009

(54) EXPRESSION OF SOLUBLE ANTIBODY FRAGMENT BY TRUNCATION OF CH1 DOMAIN

(75) Inventors: Diane Retallack, Poway, CA (US); Jon C. Mitchell, Zionsville, IN (US)

Correspondence Address: TRASKBRITT, P.C.\Dow Global Technologies Inc., PO Box 2550, SALT LAKE CITY, UT, 84110, US

(73) Assignee: DOW GLOBAL TECHNOLOGIES INC., Midland, MI (US)

(21) Appl. No.: 12/135,071

(22) Filed: Jun. 6, 2008

Related U.S. Application Data
(60) Provisional application No. 60/942,997, filed on Jun. 8, 2007.

(51) Int. Cl. C12P 21/00; C12N 15/64; C12N 1/21

(52) U.S. Cl. 435/71.2; 435/320.1; 435/252.34

(57) ABSTRACT

Improved expression of active antibody fragments (Fabs) is achieved by truncating a heavy chain constant region. Truncation of the C_{H}1 domain of a Fab fragment can increase yield of soluble active antibody fragment in Pseudomonas fluorescens. Another embodiment of the invention includes secretion of the light chain and a fragment of the heavy chain with various C-termini (e.g., V_{H}-C_{H}1 truncated to different lengths). The truncated C_{H}1 region can be used as a scaffold to create other Fabs. Also included is truncation of the kappa light chain and/or lambda light chain domains of a Fab fragment. The invention also includes expression of Fab fragments fused to other peptides or molecules (e.g., toxins, proteins, peptides, enzymes, etc.).

### CROSS-REFERENCE TO RELATED APPLICATION

This application is a utility conversion of U.S. Provisional Patent Application Ser. No. 60/942,997, filed Jun. 8, 2007, titled “EXPRESSION OF SOLUBLE ANTIBODY FRAGMENT BY TRUNCATION OF CH1 DOMAIN.”

### FIELD OF THE INVENTION

The present invention relates generally to production of soluble active antibody fragments and, more specifically, to expression of active antibody fragments (Fabs) by truncating the heavy chain constant regions thereof.

### BACKGROUND OF THE INVENTION

Natural immunoglobulins have been known for many years, as have the various fragments thereof, such as the Fab, (Fab′), sub2, and Fc fragments, which can be derived by enzymatic cleavage. Natural immunoglobulins comprise a generally Y-shaped molecule having an antigen-binding site at the end of each arm. The remainder of the structure, and particularly the stem of the Y, mediates the effector functions associated with immunoglobulins.

At the junction of the arms of the Y-shaped molecule, there is an area known as the hinge region. In this region there are, depending on the class of the antibody, at least two inter-heavy chain disulphide bonds. These disulphide bonds are responsible for holding together the two parts of the complete antibody molecule. In a Fab fragment, the hinge region has been enzymatically separated from the antigen banding region. Thus, the Fab fragment comprises a light chain/truncated heavy chain dimer.

Natural immunoglobulins and their fragments have been used in diagnosis and, to a more limited extent, in therapy. However, such uses, especially in therapy, have been hindered by the polyclonal nature of natural immunoglobulins. A significant step towards the realization of the potential of immunoglobulins as therapeutic agents was the discovery of monoclonal antibodies of defined antigen specificity.

In all the work carried out so far, the hinge region, if present, in the antibody molecule or fragment has been that normally associated with the CH1 domain of the antibody molecule. It has been suggested that the hinge region be altered or mutated to produce Fab or Fab′ fragments or altered antibody molecules, or to alter the C- or N-terminal sequence of such fragments to facilitate manipulations by recombinant DNA technology.

For example, International Application WO 2005/003170 describes an antibody Fab or Fab′ fragment to which at least one effector molecule is attached. The fragment is characterized in that the heavy chain in the fragment is not covalently bonded to the light chain and both the interchain cysteine of C_{L} and the interchain cysteine of C_{H}1 have been replaced with another amino acid. International Application WO 2005/003170 describes an antibody Fab or Fab′ fragment in which the heavy chain is not covalently bonded to the light chain and two or more effector molecules are attached to the fragment. At least one of the effector molecules is attached to a cysteine in the heavy or light chain constant region.

U.S. Pat. No. 5,677,425 and International Application WO 89/01974 to Bodmer et al. describe an altered antibody molecule (AAM) having a hinge region which has a different number of cysteine residues from that found in the hinge region normally associated with the C_{H}1 domain of the antibody molecule and a process for producing the same using recombinant DNA technology.

It would be advantageous to provide improved expression of active antibody fragments, as well as improved folding and solubility. It would be further advantageous to provide methods of yielding more active antibody fragments.

### BRIEF SUMMARY OF THE INVENTION

The present invention includes improved expression of active antibody fragments (Fabs) by truncating a heavy chain constant region. Truncation can include removal of the cysteine amino acid required for disulfide bond formation with the light chain, which can result in improved expression of active Fab at both small and large scale in prokaryotes. Improved expression of Fab fragments in microbial systems can improve production of therapeutics and diagnostics through use of these molecules. Additionally, a resulting truncated C_{H}1 region can be used as a scaffold tool for construction of other Fab molecules.

One embodiment of the present invention includes truncation of the C_{H}1 domain of a Fab fragment to increase yield of soluble active antibody fragment in Pseudomonas fluorescens. Another embodiment of the invention includes secretion of the light chain and a fragment of the heavy chain with various C-termini (e.g., V_{H}-C_{H}1 truncated to different lengths). Alternatively, the truncated C_{H}1 region can be used as a scaffold to create other Fabs (e.g., through combination with alternate V_{H} and light chains). Another embodiment of the invention includes truncation of the kappa light chain and/or lambda light chain domains of a Fab fragment. Yet another embodiment of the invention includes expression or secretion of the kappa and/or lambda light chain(s).

In another aspect, the invention includes expression of Fab fragments fused to other peptides or molecules (e.g., toxins, proteins, peptides, enzymes, etc.).

### BRIEF DESCRIPTION OF THE SEVERAL VIEWS OF THE DRAWINGS

FIG. 1 illustrates plasmids designed to express 3 Gal2 Fab variants (pDOW1196, pDOW3716 and pDOW3717);

FIG. 2 shows alignment of tested Fab heavy chain C-termini;

FIGS. 3-5 show Fab coding regions for three constructs;

FIG. 6 shows analysis of Fab expression at shake flask scale according to one embodiment of the invention;

FIG. 7 shows Western analysis of Fab expression at shake flask scale according to one embodiment of the invention;

FIG. 8 shows ELISA analysis of shake flask scale according to one embodiment of the invention;

FIG. 9 shows Activity of Gal2 Fab expressed at fermentation scale to one embodiment of the invention;

FIG. 10 shows analysis results for design of experiments illustrating responses (soluble active protein) according to one embodiment of the invention; and

FIG. 11 shows ELISA analysis of Fab strains over-expressing FrnE according to one embodiment of the invention.

### DETAILED DESCRIPTION OF THE INVENTION

As used herein, the phrase “a cultured cell into which has been introduced an expression vector” includes cells that have been physically manipulated to contain the vector, as well as progeny of the manipulated cells when the progeny also contain the vector.

The terms “amino-terminal” (or “N-terminal”) and “carboxyl-terminal” (or “C-terminal”) are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

The term “antibody” is used herein in the broadest sense and specifically covers single monoclonal antibodies, immunoglobulin chains or fragments thereof, which react immunologically with a corresponding polypeptide, such as IFN-γ or an IFN-γ receptor as well as anti-IFN-γ and anti-IFN-γ receptor antibody compositions with polyepitopic specificity, which have such properties.

The term “monoclonal antibody” (mAb) as used herein refers to an antibody (as hereinabove defined) obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins.

The light chains of antibodies (immunoglobulins) from any vertebrate species can be assigned to one of two clearly distinct types, called kappa and lambda, based on the amino acid sequences of their constant domains.

The term “corresponding to,” when applied to positions of amino acid residues in sequences, means corresponding positions in a plurality of sequences when the sequences are optimally aligned.

The term “expression vector” is used to denote a DNA molecule, linear or circular, that comprises a segment encoding a polypeptide of interest operably linked to additional segments that provide for its transcription. Such additional segments include promoter and terminator sequences, and can also include one or more origins of replication, one or more selectable markers, an enhancer, etc. Expression vectors are generally derived from plasmid or viral DNA, or may contain elements of both.

An “immunoglobulin” is a serum protein which functions as an antibody in a vertebrate organism. Five classes of “immunoglobulin,” or antibody, protein (IgG, IgA, IgM, IgD, and IgE) have been identified in higher vertebrates. IgG comprises the major class; it normally exists as the second most abundant protein found in plasma. In humans, IgG consists of four subclasses, designated IgG1, IgG2, IgG3, and IgG4. The heavy chain constant regions of the IgG class are identified with the Greek symbol γ. For example, immunoglobulins of the IgG1 subclass contain a γ1 heavy chain constant region. Each immunoglobulin heavy chain possesses a constant region that consists of constant region protein domains (CH1, hinge, CH2, and CH3) that are essentially invariant for a given subclass in a species. DNA sequences encoding human and non-human immunoglobulin chains are known in the art. See, for example, Ellison et al., DNA 1:11-18, 1981; Ellison et al., Nucleic Acids Res. 10:4071-4079, 1982; Kenten et al., Proc. Natl. Acad. Sci. USA 79:6661-6665, 1982; Seno et al., Nuc. Acids Res. 11:719-726, 1983; Riechmann et al., Nature 332:323-327, 1988; Amster et al., Nuc. Acids Res. 8:2055-2065, 1980; Rusconi and Kohler, Nature 314:330-334, 1985; Boss et al., Nuc. Acids Res. 12:3791-3806, 1984; Bothwell et al., Nature 298:380-382, 1982; van der Loo et al., Immunogenetics 42:333-341, 1995; Karlin et al., J. Mol. Evol. 22:195-208, 1985; Kindsvogel et al., DNA 1:335-343, 1982; Breiner et al., Gene 18:165-174, 1982; Kondo et al., Eur. J. Immunol. 23:245-249, 1993; and GenBank Accession No. J00228. For a review of immunoglobulin structure and function see Putnam, The Plasma Proteins, Vol V, Academic Press, Inc., 49-140, 1987; and Padlan, Mol. Immunol. 31:169-217, 1994.

The term “immunoglobulin CH1 domain” or “CH1” denotes a wild-type immunoglobulin heavy chain CH1 constant domain or a variant thereof, wherein the variant folds into the higher order structure characteristic of native immunoglobulin heavy chain constant domains (two twisted β sheets stabilized by a single disulfide bond; see, for example, Amzel and Poljak, Annu. Rev. Immunol. 48:961-997, 1979) and is capable of dimerizing with an immunoglobulin light chain constant domain.

An “immunoglobulin hinge” or “hinge” is that portion of an immunoglobulin heavy chain connecting the variable and CH1 domains.

The term “light chain <<kappa>> or <<lambda>> constant region” denotes a native immunoglobulin light chain constant domain of the <<kappa>> or <<lambda>> isotype, or a variant thereof, wherein the variant folds into the higher order structure characteristic of native immunoglobulin light chains constant domains and is capable of dimerizing with an immunoglobulin CH1 domain.

“Non-covalent associations” between polypeptides or proteins include hydrogen bonding, steric interactions, hydrophobic interactions, and ionic interactions.

A “non-immunoglobulin polypeptide” is a polypeptide that is not an immunoglobulin or fragment of an immunoglobulin. However, the term “non-immunoglobulin polypeptide” does not exclude polypeptides that contain immunoglobulin-like domains, so long as they are not themselves immunoglobulins.

“Operably linked” means that two or more entities are joined together such that they function in concert for their intended purposes. When referring to DNA segments, the phrase indicates, for example, that coding sequences are joined in the correct reading frame, and transcription initiates in the promoter and proceeds through the coding segment(s) to the terminator. When referring to polypeptides, “operably linked” includes both covalently (e.g., by disulfide bonding) and non-covalently (e.g., by hydrogen bonding, hydrophobic interactions, or salt-bridge interactions) linked sequences, wherein the desired function(s) of the sequences are retained.

A “polynucleotide” is a single- or double-stranded polymer of deoxyribonucleotide or ribonucleotide bases read from the 5′ to the 3′ end. Polynucleotides include RNA and DNA, and may be isolated from natural sources, synthesized in vitro, or prepared from a combination of natural and synthetic molecules. Sizes of polynucleotides are expressed as base pairs (abbreviated “bp”), nucleotides (“nt”), or kilobases (“kb”). Where the context allows, the latter two terms may describe polynucleotides that are single-stranded or double-stranded. When the term is applied to double-stranded molecules it is used to denote overall length and will be understood to be equivalent to the term “base pairs.” It will be recognized by those skilled in the art that the two strands of a double-stranded polynucleotide may differ slightly in length and that the ends thereof may be staggered as a result of enzymatic cleavage; thus all nucleotides within a double-stranded polynucleotide molecule may not be paired. Such unpaired ends will in general not exceed 20 nt in length.

A “polypeptide” is a polymer of amino acid residues joined by peptide bonds, whether produced naturally or synthetically. Polypeptides of less than about 10 amino acid residues are commonly referred to as “peptides.”

The term “promoter” is used herein for its art-recognized meaning to denote a portion of a gene containing DNA sequences that provide for the binding of RNA polymerase and initiation of transcription. Promoter sequences are commonly, but not always, found in the 5′ non-coding regions of genes.

A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

A “segment” is a portion of a larger molecule (e.g., polynucleotide or polypeptide) having specified attributes. For example, a DNA segment encoding a specified polypeptide is a portion of a longer DNA molecule, such as a plasmid or plasmid fragment, that, when read from the 5′ to the 3′ direction, encodes the sequence of amino acids of the specified polypeptide.

The present invention includes improved expression of active antibody fragments (Fabs) by truncating the heavy chain constant regions (C_{H}1). Truncation can include removal of the cysteine amino acid required for disulfide bond formation with the light chain, which can result in improved expression of active Fab at both small (e.g., 0.5 L) and large (e.g., 20 L fermentation) scale in prokaryotes, such as P. fluorescens. Improved expression of Fab fragments in P. fluorescens and/or other microbial systems can provide an advantage in production of therapeutics and diagnostics through use of these molecules. Improved soluble expression of Fab can also enable identification of Fabs that recognize specific antigens in high throughput (HTP) format. Additionally, a resulting truncated CH1 region can be used as a scaffold tool for construction of other Fab molecules.

In contrast to other known methods, the present invention does not rely on mutating one or more cysteine residue(s) in the hinge region, but rather truncating the CH1 region of an IgG1 to up to five amino acids upstream of the cys involved in interchain disulfide between the heavy and light chain. The truncated CH1 can provide improved expression/folding/solubility and yield more active FAb as measured by ELISA. This CH1 region can be used as a scaffold tool to build other antibody fragments.

More specifically, the CH1 region is truncated by deleting the cysteine responsible for heavy-light chain disulfide linkage. Optionally, the number of amino acids deleted beyond the cysteine residue may vary. Optionally, an additional 1, 2, 3, 4, 5, 6, 7, 8, 9, 10, 11, 12, 13, 14, 15, 16, 17, 18, 19, 20, 25, 30, 35, 40, 45, 50 and up to 100 amino acids upstream or downstream of the cysteine can be deleted. The truncated CH region can be fused to any variable region and paired with an appropriate light chain to produce an antibody fragment that is expressed with higher solubility in prokaryotes, such as P. fluorescens, or other suitable expression systems.

In a particular embodiment of the invention, the cysteine residue in CH1 that is responsible for interchain disulfide is deleted and the four amino acids upstream of the cysteine are retained. In alternative embodiments, a greater region of CH1 can be deleted, including the four amino acids upstream of the cysteine. Expression of the CH1 region (with or without alternate variable regions) in other prokaryotic systems can be performed.

The Fab of the present invention will, in general, be capable of selectively binding to an antigen. The antigen may be any cell-associated antigen, for example, a cell surface antigen on cells such as bacterial cells, yeast cells, T-cells, endothelial cells or tumor cells, or it may be a soluble antigen. Antigens may also be any medically relevant antigen, such as those antigens unregulated during disease or infection, for example, receptors and/or their corresponding ligands. Particular examples of cell surface antigens include adhesion molecules, for example, integrins such as pi integrins, e.g., VLA-4, E selectin, Pi selectin or L-selectin, CD2, CD3, CD4, CD5, CD7, CD8, CD1 1a, CD1 1b, CD18, CD19, CD20, CD23, CD25, CD33, CD38, CD40, CD45, CDW52, CD69, carcinoembryonic antigen (CEA), human milk fat globulin (HMFG1 and 2), MHC Class I and MHC Class II antigens, and VEGF, and where appropriate, receptors thereof. Soluble antigens include; interleukins, such as IL-1, IL-2, IL-3, IL-4, IL-5, IL-6, IL-8, IL-12, IL-16 or IL-17, viral antigens for example respiratory syncytial virus or cytomegalovirus antigens, immunoglobulins, such as IgE, interferons, such as interferon oc, interferon 8 or interferon, tumor necrosis factor-oc, tumor necrosis factor-B, colony stimulating factors such as G-CSF or GM-CSF, and platelet derived growth factors, such as PDGF-oc, and PDGF-p, and where appropriate, receptors thereof.

According to one aspect of the invention, a collection of antibody fragments (Fabs) directed against a selected target can be cloned and expressed in a prokaryote (e.g., P. fluorescens) having variable C-termini. The variable region of the Fabs can be derived from a single chain antibody. The heavy and light chains can then be cloned as a single operon transcribed from a plasmid promoter (e.g., Ptac promoter of plasmid pDOW1169, as described hereafter). The heavy chain fragment can then be amplified from the plasmid such that it includes the coding region from a pbp secretion signal sequence through VH and CH1. Following the CH1 coding region, three in-frame translational stop signals can be engineered along with an XbaI site. The light chain can then be amplified, including the pbp secretion signal, and cloned into the XbaI and XhoI sites.

Another embodiment of the invention includes secretion of the light chain and a fragment of the heavy chain with various C-termini (e.g., V_{H}-C_{H}1 truncated to different lengths). Alternatively, the truncated C_{H}1 region can be used as a scaffold to create other Fabs (e.g., through combination with alternate V_{H} and light chains).

In another aspect, the invention includes expression of the aforementioned antibody fragments fused to other peptides or molecules. Molecules may be attached to antibody fragments by a number of different methods, including through aldehyde sugars or more commonly through any available amino acid side-chain or terminal amino acid functional group located in the antibody fragment, for example any free amino, imino, thiol7 hydroxyl or carboxyl group. The site of attachment of effecter molecules can be either random or site specific.

Random attachment is often achieved through amino acids, such as lysine, and this results in effecter molecules being attached at a number of sites throughout the antibody fragment depending on the position of the lysines.

Site specific attachment of molecules can be achieved by attachment to cysteine residues, since such residues are relatively uncommon in antibody fragments. Antibody hinges are popular regions for site specific attachment since these contain cysteine residues and are remote from other regions of the antibody likely to be involved in antigen binding. Suitable hinges either occur naturally in the fragment or may be created using recombinant DNA techniques (See, for example, U.S. Pat. No. 5,677,425; WO98/25971; Leong et al., 2001 Cytokine, 16, 106-119; Chapman et al., 1999 Nature Biotechnology, 17, 780-783). Alternatively, site specific cysteines may be engineered into the antibody fragment for example to create surface exposed cysteine(s) (See U.S. Pat. No. 5,219,996).

Suitable molecules for use with the invention include, for example, antineoplastic agents, drugs, toxins (such as enzymatically active toxins of bacterial or plant origin and fragments thereof, e.g., ricin and fragments thereof), biologically active proteins, for example enzymes, other antibody or antibody fragments, synthetic or naturally occurring polymers, nucleic acids and fragments thereof, e.g., DNA, RNA and fragments thereof, radionuclides, particularly radioiodide, radioisotopes, chelated metals, nanoparticles and reporter groups, such as fluorescent compounds or compounds which may be detected by NMR or ESR spectroscopy.

Particular antineoplastic agents include, for example, cytotoxic and cytostatic agents for example alkylating agents, such as nitrogen mustards (e.g., chlorambucil, melphalan, mechlorethamine, cyclosphophamide, or uracil mustard) and derivatives thereof, triethylenephosphoramide, triethylenethiophosphor-amide, busulphan, or cisplatin; antimetab olites, such as methotrex ate, fluorouracil, floxuri dine, cytarabine, mercaptopurine, thioguanine, fluoroacetic acid, or fluorocitric acid, antibiotics, such as bleomycins (e.g., I bleomycin sulphate), doxorubicin, daunorubicin, mitomycins (e.g., mitomycin C), actionmycins (e.g., dactinomycin) plicamyin, calichaemicin and derivatives thereof, or I esperamicin and derivatives thereof; mitotic inhibitors, such as etoposide, vincristine or vinblastine and derivatives thereof; alkaloids such as ellipticine; polyols such as taxicin-I or i taxicin-II; hormones, such as androgens (e.g., dromostanolone or testolactone), progestins (e.g., megestrol acetate or medroxyprogesterone acetate), estrogens (e.g., dimethylstilbestrol diphosphate, polyestradiol phosphate or estramustine phosphate) or antiestrogens (e.g., tamoxifen); anthraquinones, such as mitoxantrone, ureas, such as hydroxyurea; hydrazines, such as procarbazine; or imidazoles, such as dacarbazine. Suitable chelated metals include, for example, chelates of di- or tripositive metals having a coordination number from 2 to 8 inclusive.

Suitable molecules for use with the invention also include proteins, peptides and enzymes. Enzymes of interest include, but are not limited to, proteolytic enzymes, hydrolases, lyases, isomerases, transferases. Proteins, polypeptides and peptides of interest include, but are not limited to, immunoglobulins, toxins such as abrin, ricin A, pseudomonas exotoxin, or diphtheria toxin, a protein such as insulin, tumor necrosis factor, oc-interferon, p-interferon, nerve growth factor, platelet derived growth factor or tissue plasminogen activator, a thrombotic agent or an anti-angiogenic agent, e.g., angiostatin or endostatin, or, a biological response modifier such as a lymphokine, interleukin-1 (IL-1), interleukin-2 (IL-2), interleukin-6 (IL-6), granulocyte macrophage colony stimulating factor (GM-CSF), granulocyte colony I stimulating factor (G-CSF), nerve growth factor (NGF) or other growth factor and immunoglobulins.

Other molecules may include detectable substances useful, for example, in diagnosis. Examples of detectable substances include various enzymes, prosthetic groups, fluorescent materials, luminescent materials, bioluminescent materials, radioactive nuclides, positron emitting metals (for use in positron emission tomography), and nonradioactive paramagnetic metal ions. Suitable enzymes include horseradish peroxidase, alkaline phosphatase, beta-galactosidase, or acetylcholinesterase; suitable prosthetic groups include streptavidin, avidin and biotin; suitable fluorescent materials include umbelliferone, fluorescein, fluorescein isothiocyanate, rhodamine, dichlorotriazinylamine fluorescein, dansyl chloride and phycoerythrin; suitable luminescent materials include luminol; suitable bioluminescent materials include luciferase, luciferin, and acquorin; and suitable radioactive nuclides include i25I, i3iI, iIn and 99Tc.

Synthetic or naturally occurring polymers for use as molecules include, for example, optionally substituted straight or branched chain polyalkylene, polyalkenylene, or polyoxyalkylene polymers or branched or unbranched polysaccharides, e.g., a homo- or hetero-polysaccharide such as lactose, amylose, dextran or glycogen. Particular optional substituents which may be present on the above-mentioned synthetic polymers include one or more hydroxy, methyl or methoxy groups. Particular examples of synthetic polymers include optionally substituted straight or branched chain poly(ethyleneglycol), poly(propyleneglycol), poly(vinylalcohol) or derivatives thereof, especially optionally substituted poly(ethyleneglycol) such as methoxypoly(ethyleneglycol) I or derivatives thereof. Particularly preferred polymers include a polyalkylene polymer, such as a poly(ethyleneglycol) or, especially, a methoxypoly(ethyleneglycol) or a derivative thereof, and especially with a molecular weight in the range from about 10,000 Da to about 40,000 Da.

“Derivatives” as used herein is intended to include reactive derivatives, for example thiol-selective reactive groups such as an α-halocaraboxylic acid or ester, e.g., iodoacetamide, an imide, e.g., maleimide, a vinyl sulphone or disulphide malemides and the like.

In one example, the molecules of the present invention may be attached to the protein through any available amino acid side-chain or terminal amino acid functional group located in the Fab, for example any free amino, imino, thiol, hydroxyl or carboxyl group. Such amino acids may occur naturally in the Fab or may be engineered into the fragment using recombinant DNA methods.

In a particular aspect of the present invention, at least one of the molecules attached to the antibody fragment is a polymer molecule, preferably PEG or a derivative thereof.

The present invention also includes a host cell expressing the antibody Fab fragment intermediate (e.g., CH1 truncated fragment or a VH-CH1 truncated fragment). Any suitable host cell/vector system may be used for the expression of the DNA sequences encoding the antibody Fab intermediate of the present invention. Prokaryotic host cells, including strains of the bacteria Pseudomonas, E. coli, Bacillus and other genera are also useful host cells within the present invention. Techniques for transforming these hosts and expressing foreign DNA sequences cloned therein are well known in the art. When expressing a polypeptide fusion in bacteria, the polypeptide may be retained in the cytoplasm as insoluble granules, or it may be directed to the periplasmic space by a bacterial secretion sequence. The protein is recovered from the cell as an aqueous extract in, for example, phosphate buffered saline. To capture the protein of interest, the extract is applied directly to a chromatographic medium, such as an immobilized antibody or heparin-Sepharose column. Secreted polypeptides can be recovered from the periplasmic space in a soluble and functional form by disrupting the cells (by, for example, sonication or osmotic shock) and recovering the protein, thereby obviating the need for denaturation and refolding. See, for example, Lu et al., J. Immunol. Meth. 267:213-226, 2002.

Transformed or transfected host cells can be cultured according to conventional procedures in a culture medium containing nutrients and other components required for the growth of the chosen host cells. A variety of suitable media, including defined media and complex media, are known in the art and generally include a carbon source, a nitrogen source, essential amino acids, vitamins and minerals. Media may also contain such components as growth factors or serum, as required. The growth medium will generally select for cells containing the exogenously added DNA by, for example, drug selection or deficiency in an essential nutrient which is complemented by the selectable marker carried on the expression vector or co-transfected into the host cell.

The antibody fragments according to the invention may be useful in the detection or treatment of a number of diseases or disorders. Such diseases or disorders may include those described under the general heading of infectious disease, e.g., bacterial infection; fungal infection; inflammatory disease/autoimmunity, e.g., rheumatoid arthritis, osteoarthritis, inflammatory bowel disease; cancer; allergic/atopic disease, e.g., asthma, eczema; congenital disease, e.g., cystic fibrosis, sickle cell anemia; dermatologic disease, e.g., psoriasis; necrologic disease, e.g., multiple sclerosis; transplants, e.g., organ transplant rejection, graft-versus-host disease; and metabolic/idiopathic disease, e.g., diabetes. The antibody fragments according to the invention may be formulated for use in therapy and/or diagnosis and according to a further aspect of the invention a pharmaceutical composition comprising an antibody fragment (alone or in combination with a peptide or molecule) is provided together with one or more pharmaceutically acceptable excipients, diluents or carriers.

The present invention is explained in greater detail in the Examples that follow. These examples are intended as illustrative of the invention and are not to be taken are limiting thereof.

### EXAMPLES

### Example 1

### Construction of Fab Variants

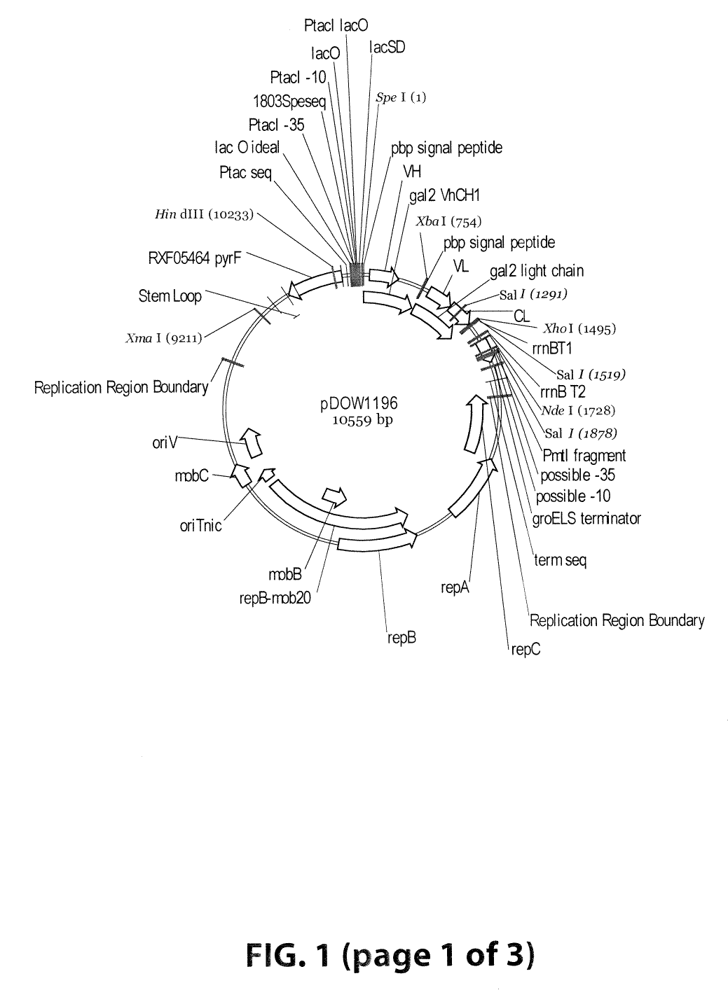

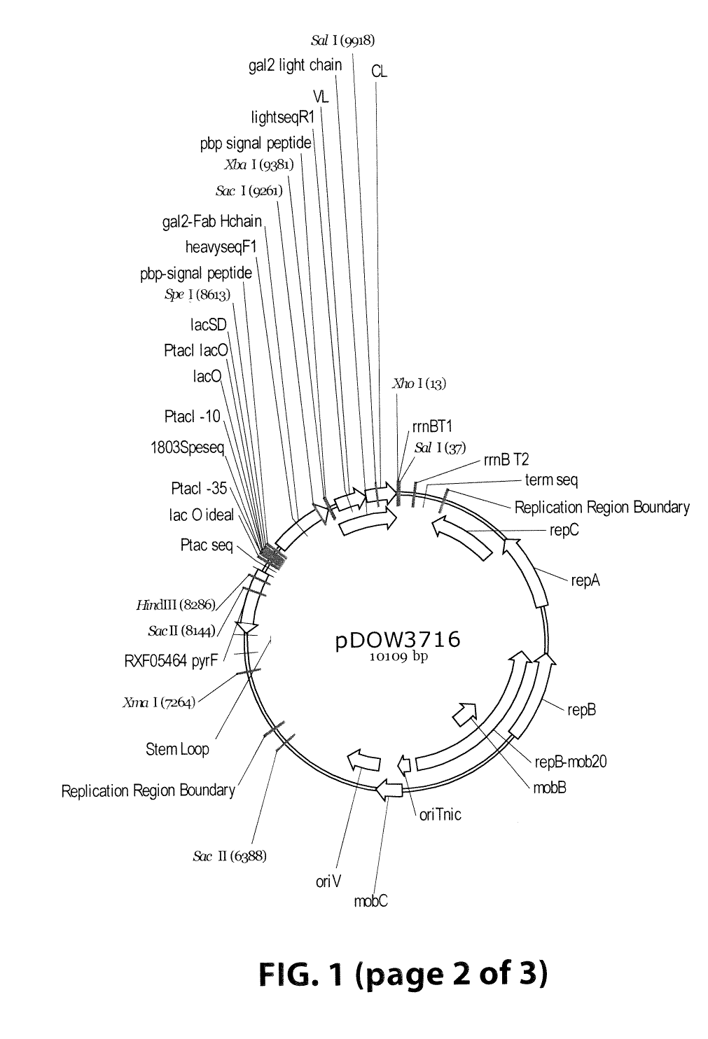

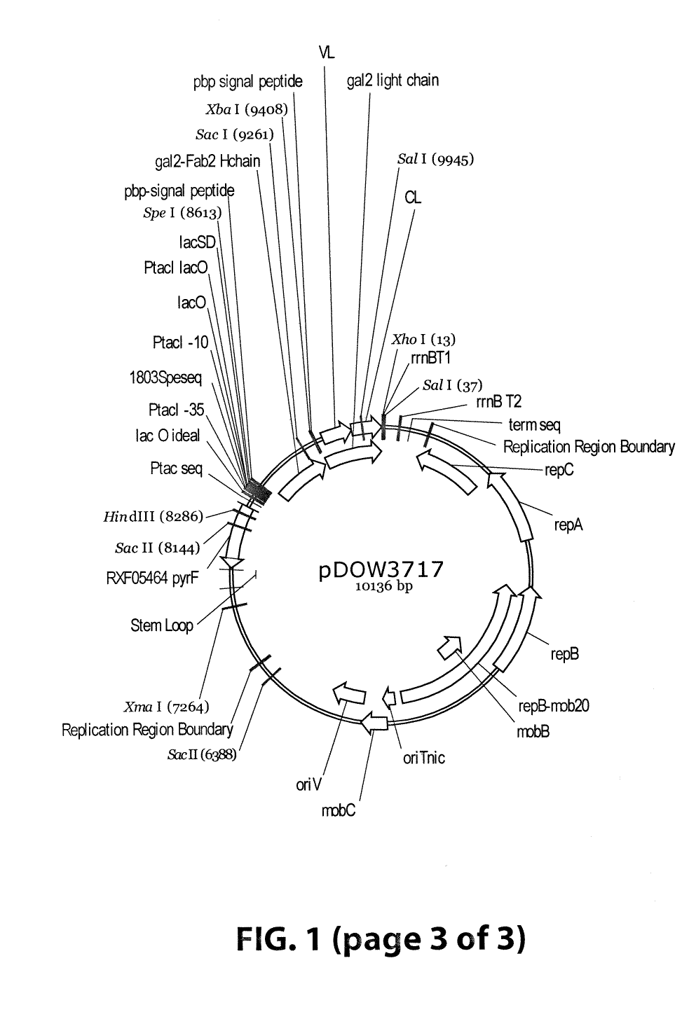

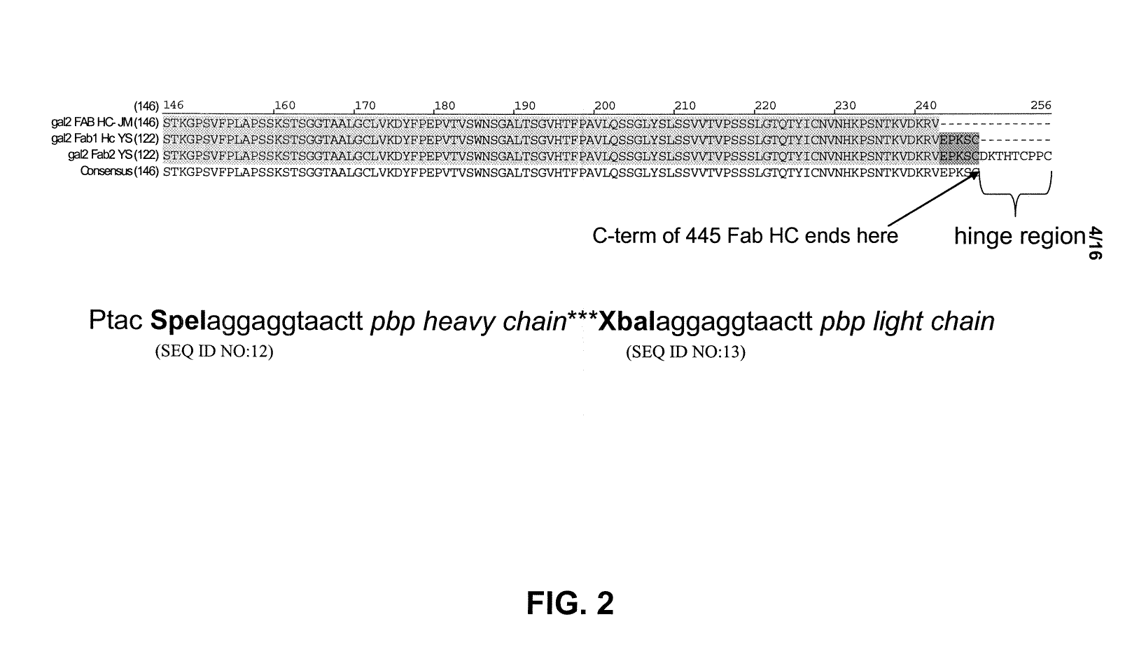

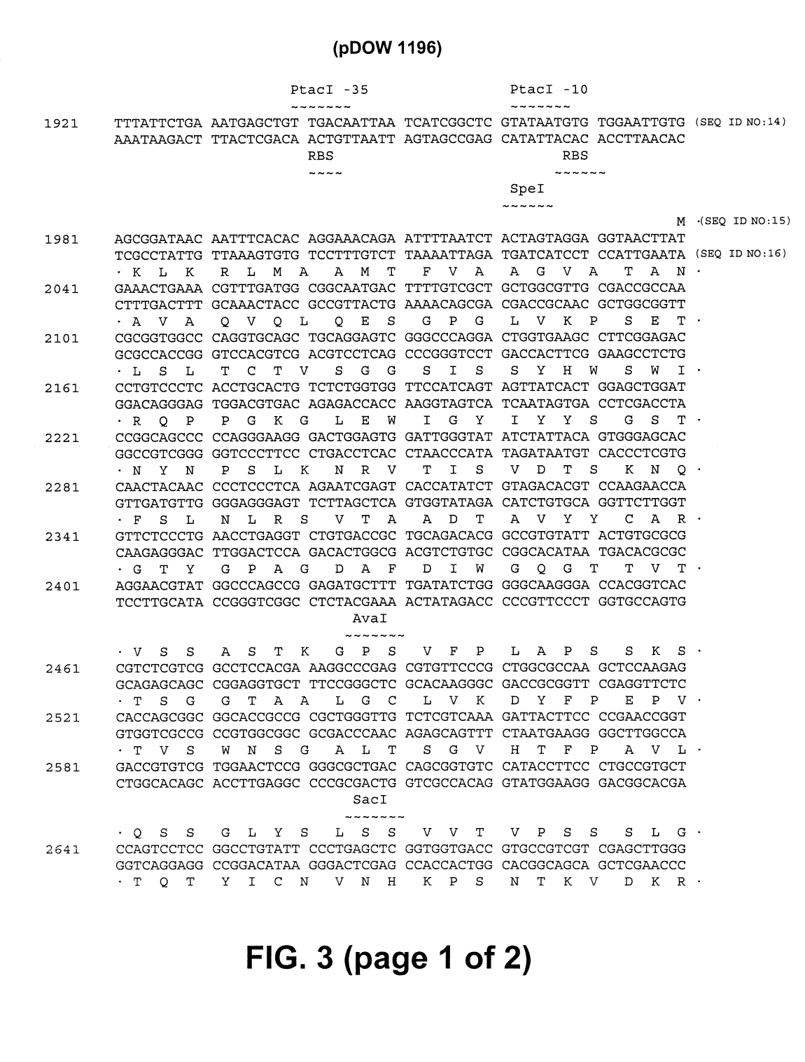

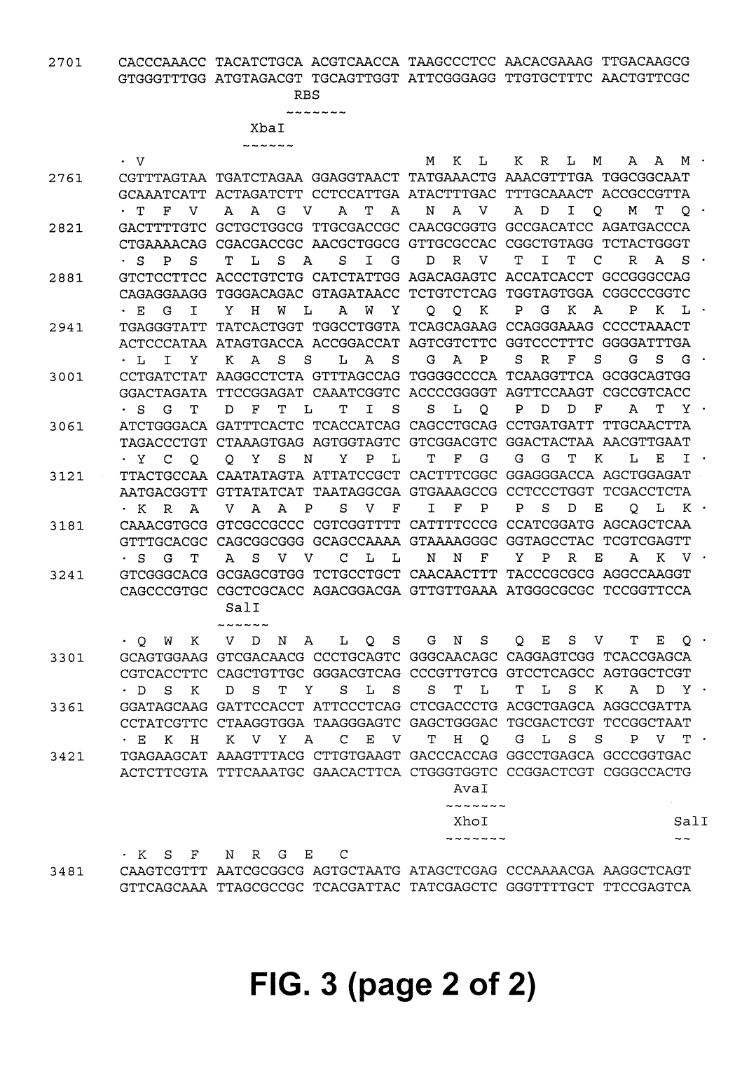

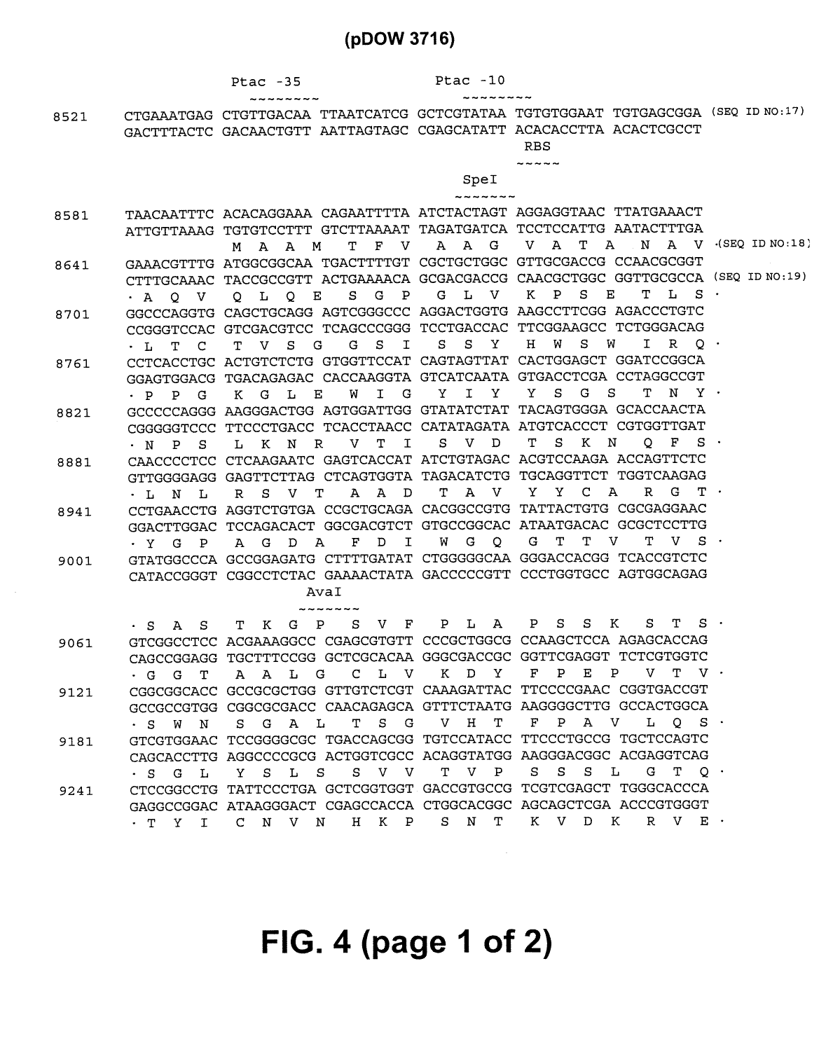

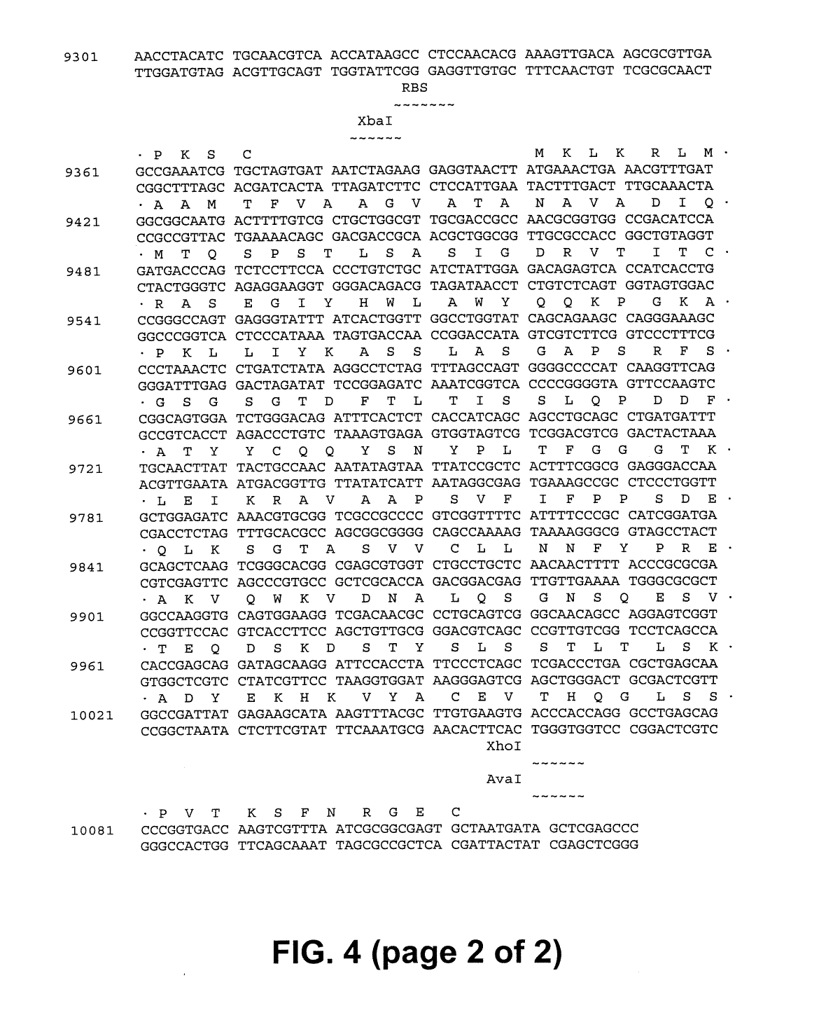

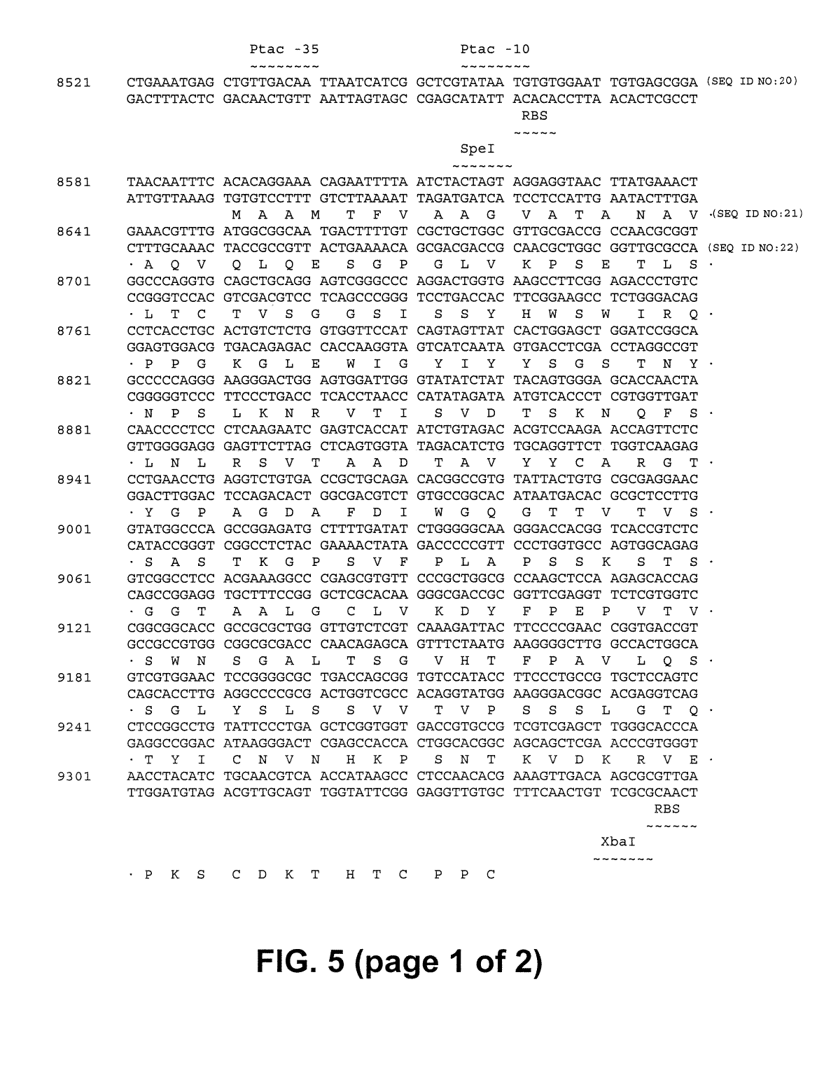

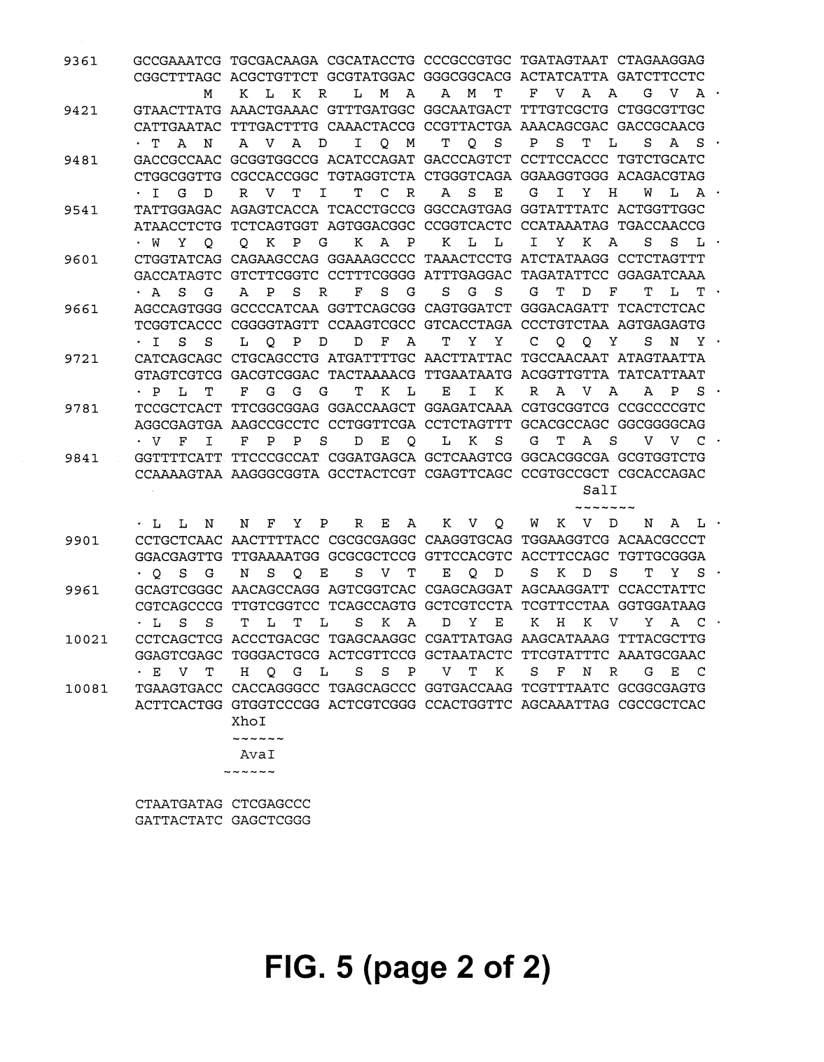

Plasmids were designed to express 3 Gal2 Fab variants (pDOW1196, pDOW3716 and pDOW3717), each with the Gal2 light chain and with Gal2 heavy chain containing CH1 regions of different lengths, as shown in FIG. 1. A comparison of the heavy chain C-termini is shown in FIG. 2. Plasmid pDOW1196 contains the shortest CH1 region, truncated 4 amino acids N-terminal to the cysteine residue involved in interstrand disulfide bond with the light chain. Plasmid pDOW3716 contains a CH1 region that extends to the cysteine required for interstrand disulfide and pDOW3717 contains a CH1 region that extends through the hinge region. The Gal2 heavy and light chains fused to the phosphate binding protein (pbp) signal sequence were amplified from the Gal2 mAB expression plasmid pDOW2788. Each heavy chain/light chain combination was cloned behind the Ptac promoter as a single operon, with the heavy chain coding sequence followed by three nonsense codons, and XbaI site, optimal ribosome binding site and 7 nucleotide space prior to the start of the pbp-light chain coding sequence. The resulting coding regions are shown in FIGS. 3-5.

### Example 2

### Construction and Expression of Gal2 Fab Variants in P. fluorescens

P. fluorescens strains DC454 (ΔpyrF lsc::lacI^{Q}) (Schneider et al. 2005) and DC572 (ΔpyrFΔproCΔbenAB ΔmtlDYZ lsc::lacI^{Q} Pmtl:frnE proC) were used as expression hosts. DC572 over-expresses the frnE homologue (RXF08657), a putative disulfide isomerase.

Fab expression plasmid construction: Standard cloning techniques were used for the construction of Fab expression plasmids (Sambrook et al. 2001). The plasmid pDOW1196 was constructed as follows. The heavy chain region of the Gal2 mAB (V. Lee et al., report in preparation) was amplified from pDOW2788 using primers gal2HC_{—}5′ (ACTAGTAGGAGGTAACTTATGAAACTGAAACGTTTGATGGC (SEQ ID NO:1)) and XbaI_VhCH1_R (TCTAGATCATTACTAAACGCGCTTGTCACCTTTCGTGTT (SEQ ID NO:2)). PCR fragments were cloned into pCR2.1TOPO (Invitrogen), transformed into E. coli Top10 and selected on LB Soy Agar Amp100 (Teknova). Plasmid prepared from transformants was screened by sequencing, and a positive cloned identified. The sequence confirmed fragment was restriction digested with SpeI and XbaI, and ligated to pDOW1173 digested with the same enzymes. DC454 was transformed with the resultant ligation and transformants were selected on M9 glucose agar (Teknova) and screened for insert by restriction digestion with SpeI and XbaI. The Gal2 light chain was amplified from pDOW2788 using primers XbaI_pbp_F (TCTAGAAGGAGGTAACTTATGAAACTGAAACGTTTGATG (SEQ ID NO:3)) and XhoI_L_R (CTCGAGCTATCATTAGCACTCGCCGCGATTAAACGACTT (SEQ ID NO:4)), and the resultant fragment cloned into pCR2.1TOPO and confirmed, as previously described. The sequence confirmed that the light chain fragment was restriction digested with XbaI and XhoI and ligated to pDOW1173+gal2 heavy chain fragment (constructed as described above) digested with the same enzymes. DC454 was transformed with the ligation mix and transformants were M9 glucose agar (Teknova) and screened for insert by restriction digestion with XbaI and XhoI.

To construct the plasmids pDOW1197 and pDOW1198, containing the pbp-gal2 heavy chain with and without the hinge region of gal2, respectively, fragments were amplified by PCR (Stratagene Cat#600600) using plasmid pDOW2787 containing gal2 monoclonal antibody (mAb) as the template. The pbp-gal2 heavy chain without the hinge region was amplified using primers gal2HC_{—}5′ (ACTAGTAGGAGGTAACTTATGAAACTGAAACGTTTGATGGCGG CAA (SEQ ID NO:5)) and CH1_rev (CGTCTAGATTATCACTAGCACGATTTCGGCTCAAC (SEQ ID NO:6)) under cycling conditions of 94° C. for two minutes, (94° C. for 30 seconds; 45° C. for 30 seconds; 70° C. for two minutes) 30×, 72° C. for ten minutes. The pbp-gal2 heavy chain with the hinge region was amplified using primers gal2HC_{—}5′ and CH1_hinge (GCTCTAGATTACTATCAGCACGGCGGGCAGGTATGC (SEQ ID NO:7)) under the same condition mentioned above. The purified PCR products were digested with restriction enzymes SpeI and XbaI, ligated into the plasmid pDOW1169 between the same restriction sites and transformed into DC454 (ΔpyrF lsc::lacIq1). Resulting transformants were sequenced and the positive clones were named pDOW1197 and pDOW1198, respectively. The XbaI—XhoI fragment from pDOW1196, containing the gal2 light chain coding region, was ligated to each pDOW1197 and pDOW1198 digested with the same enzyme. P. fluorescens DC454 was transformed with the ligation products, selected on M9 glucose agar, and positive clones were sequenced to confirm. The resultant plasmids were named pDOW3716 and pDOW3717, respectively.

Shake Flask Expression: The P. fluorescens strain DC454 or DC572 carrying each clone was analyzed by the standard Dow 1 L-scale shake-flask expression protocol. Seed cultures grown in a standard medium supplemented with 1% glucose and trace elements were used to inoculate 200 mL of defined minimal salts medium with 5% glycerol as the with a carbon source. Following an initial growth phase, expression via the Ptac promoter was induced with isopropyl-β-D-1-thiogalactopyranoside (IPTG). Where indicated, frnE was induced with 0.5% mannitol. Cultures were sampled at the time of induction (10), and at 24 hours post-induction (124). Cell density was measured by optical density at 600 nm (OD₆₀₀). The cell density was adjusted to OD₆₀₀=20, and 100 μl aliquots were centrifuged at 14000×g for five minutes. Supernatants (cell free broth) were pipetted into a new microfuge tube, then cell pellets and cell free broth samples were frozen at −80° C. for later processing.

Expression in mini-bioreactors: Strains were supplied as glycerol stocks from TDCC culture collection. Seed flasks composed of 600 ml PS/2 formula medium with glycerol were inoculated with 600 μl of thawed glycerol stock for each strain and incubated at 30° C., 300 rpm for 20 to 24 hours. Seed flask cell density was typically measured as 12 to 20 optical density (O.D.) at 575 nm on a visible light spectrometer. Seed flasks were typically 5.5 to 6.8 pH.

The DASGIP FedBatch Pro system of 500 ml small-scale fermentors was used for strain assessment and Design of Experiment procedures. Samples for analysis were taken post induction at 0, 8, 16 and 24 hours. Samples were immediately analyzed for O.D. and pH. Aliquots of 0.100 g were removed, centrifuged ten minutes in a microcentrifuge at maximum velocity and the supernatant was removed by pipet. The pellet and the supernatant (cell free broth or CFB) were saved separately at −20° C. until analysis by Western Blotting and/or ELISA.

### Example 3

### SDS-PAGE, Western and ELISA Analyses

Soluble and insoluble fractions from shake flask samples were generated using Easy Lyse (Epicentre Technologies). The frozen pellet was resuspended and diluted 1:4 in lysis buffer and incubated with shaking at room temperature for 30 minutes. The lysate was centrifuged at 14,000 rpm for 20 minutes (4° C.) and the supernatant removed. The supernatant was saved as the soluble fraction. The pellet (insoluble fraction) was then resuspended in an equal volume of lysis buffer and resuspended by pipetting up and down. Cell free broth samples were thawed and used at full strength. Samples were mixed 1:1 with 2× Laemmli sample buffer containing β-mercaptoethanol (BioRad cat# 161-0737) and boiled for five minutes prior to loading 15 μL on a Bio-Rad Criterion 12% Criterion XT gel (BioRad) and electrophoresis in the recommended 1×MES buffer (BioRad). Gels were stained with Simply Blue Safe Stain (Invitrogen cat# LC6060) according to the manufacturer's protocol and imaged using the Alpha Innotech Imaging system. Western analysis was performed as described (RP-MB-015). For shake flask samples, 10 μL 20OD normalized sample was loaded. For fermentation samples 10 μl of a 40× dilution of the soluble and insoluble fractions or 5 μl of a 20× dilution of the CFB fractions were loaded onto a 12% Bis-Tris polyacrylamide gel. Anti-human kappa light chain horseradish peroxidase HRP conjugate (Sigma A7164) diluted 1:5000 was used as detection antibody.

ELISA: The antibody Goat anti-human Kappa conjugated to horseradish peroxidase (Sigma A7164) was used at 1:30,000 dilution in blocking solution consisting of PBS with 2% w/v skim milk. The wells of an ELISA plate were filled with 200 μl of 10 μg/ml β-galactosidase solution and incubated at room temperature overnight to prepare a test plate. The Gal2 standard was prepared by diluting Protein A, purified (050628B) 100 fold in the above blocking solution, then serially diluted seven times with a four-fold dilution each time. The eight standard dilutions were transferred in duplicate to separate wells of the prepared test plate. The fermentation samples, prepared as described above, were serially diluted separately three times, five-fold at each dilution, to equal four different dilutions. The dilutions were transferred, in duplicate, to separate wells of the prepared test plate. The completed test plate was incubated at room temperature for two hours, then washed with PBST. The kappa antibody was added to all of the wells and incubated at room temperature for two hours. The plate was then washed with PBST. After washing, the calorimetric substrate TMB was added to all wells and the plate was incubated at room temperature eight to ten minutes. After color development was complete, 2N H2SO4 was added to stop the reaction. The plate was then read with a UV spectrophotometric plate reader.

Fermentation Design of Experiment: A full-factorial screening design with two factors and a mid-point was designed and analyzed using SAS JMP software, version 6.0. The selected best strain of Gal2Fab (DC536) was inoculated into ten separate DASGIP fermentors and grown with the predetermined 2×2×2 factors (and duplicate mid-points). Cultures were grown to the target O.D. of 180 (±18) then induced at the pre-determined conditions as indicated in the following table. Order of experimental conditions was randomized by the JMP program. At harvest, 24 hours post induction, samples were taken, processed an analyzed as described above. The ELISA values for the soluble fractions were recorded in the JMP statistical file as effect and the model for effect screening was run with all factors modeled separately and as second order interactions.

### Example 4

### Expression of Fab Variants at Shake Flask Scale

Each plasmid was transformed into wild-type P. fluorescens (DC454) as well as a P. fluorescens strain that over-expresses the putative disulfide isomerase FrnE (DC572). Thus far, over-expression of the disulfide oxido-reductase and isomerase DsbA and DsbC proteins has not shown improved solubility of monoclonal antibodies, antibody fragments or other disulfide bonded proteins tested (Retallack et al. 2006a; Retallack et al. 2006b; Shao et al. 2006; Coleman, Schneider et al. 2007). DC454 and DC572 transformed with each Fab construct, pDOW1196 (truncated CH1), pDOW3716 (Fab) and pDOW37117 (Fab+hinge) (see Table 1) was subjected to small scale (shake flask) expression analysis.

TABLE 1
Strains Constructed for this study

| Plasmid Number | DC454 host strain | DC572 host strain |
|---|---|---|
| pDOW1196 | DC536 | DC593 |
| pDOW3716 | DC589 | DC591 |
| pDOW3717 | DC590 | DC592 |

All strains grew to approximately 15 ODs at the time of induction. The heavy and light chain genes, along with the frnE gene in the DC572 host, were induced with IPTG and mannitol respectively following 24 hours of growth. Cell density increased to 16-20 ODs for all strains except DC572 carrying pDOW3717 (DC592), which decreased slightly in optical density.

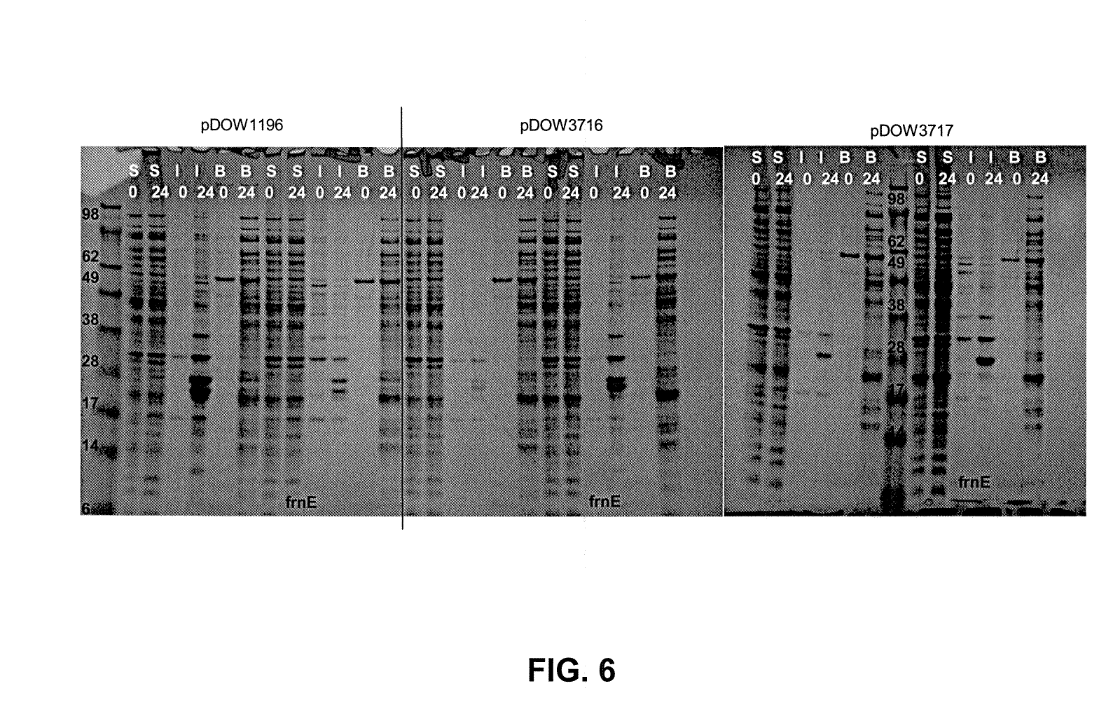

The truncated CH1 product heavy chain (pDOW1196) was expected to be 25.4 kDa without secretion leader processing, 23 kDa with leader processing. The heavy chain product of encoded by pDOW3716 was expected to be 26 kDa without leader processing and 23.5 kDa with leader processing; the heavy chain product of encoded by pDOW3717 was expected to be 26.7 kDa without leader processing and 24.5 kDa with leader processing. The light chain was expected to be 25.7 kDa without leader processing and 23.3 kDa with leader processing. Reducing SDS-PAGE analysis revealed expression of predominantly insoluble heavy and/or light chain from both the pDOW1196 and pDOW3717 constructs, as shown in FIG. 6. Over-expression of frnE appeared to reduce the amount of induced insoluble protein expressed from pDOW1196, whereas more induced insoluble protein was observed in frnE over-expressing strains carrying pDOW3716 or pDOW3717. Western analysis was performed to detect light chain expression using an anti-kappa antibody. As shown in FIG. 7, samples run under reducing conditions showed predominantly processed light chain, although a small amount of unprocessed soluble light chain was expressed from the DC454/pDOW1196 strain (FIG. 7A). Western analyses run under non-reducing conditions (FIG. 7B) indicated that dimers containing light chain were formed. In the pDOW1196 containing strain (truncated CH1), both light chain monomer as well as an intermediate sized multimer were detected. Moreover, light chain was detected in only the soluble and cell free broth fractions of the pDOW1196-containing strain under non-reducing conditions, although a significant amounts of insoluble light chain were detected under reducing conditions in both SDS-PAGE and Western analyses. However, insoluble light chain was detected associated with a multimer expressed from strains carrying pDOW3716 and pDOW3717 (FIG. 7B). In the case of strains carrying pDOW3717 (CH1 with hinge) light chain was detected as insoluble protein in the wild-type host. However, light chain was detected in the soluble and cell free broth fractions as well in the FrnE over-expression host, as both monomer under reducing conditions and multimer under non-reducing conditions. Moreover, an increased amount of insoluble light chain in the FrnE over-expression host was seen compared to that detected in the wild-type host, as shown in FIG. 7.

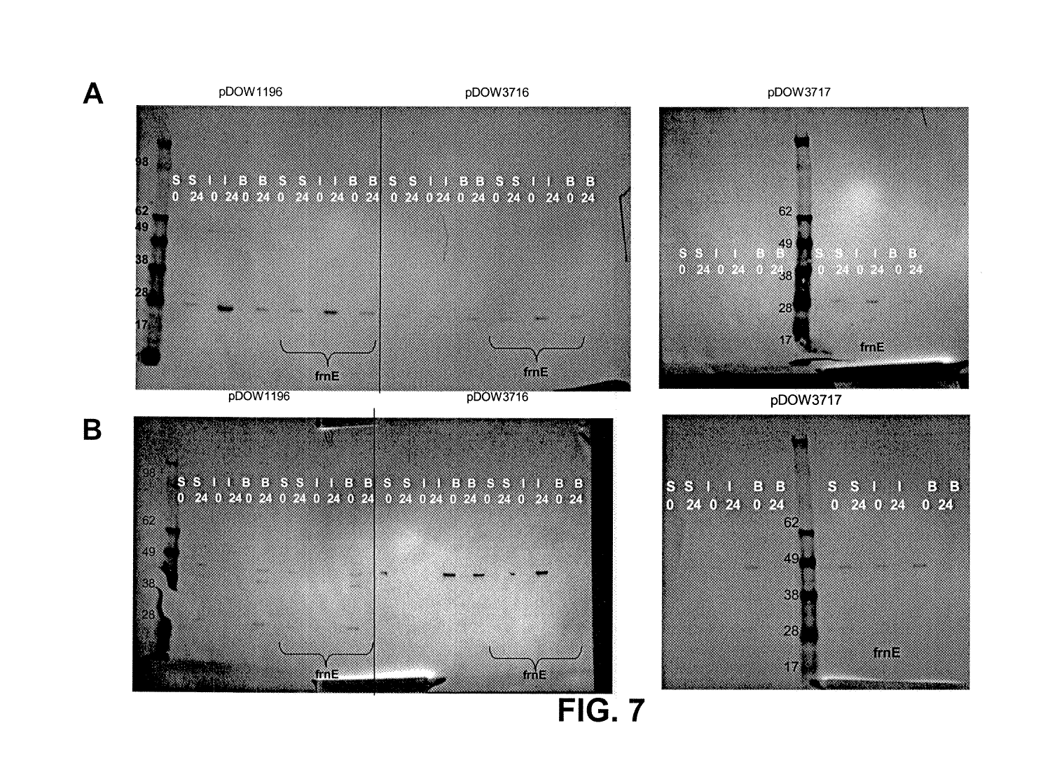

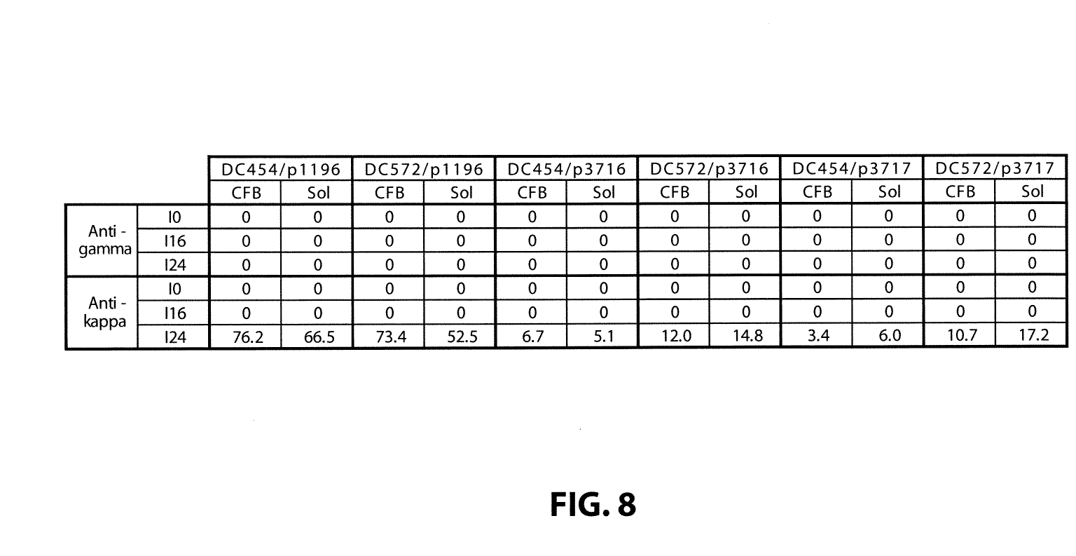

ELISA was performed to assay the level of active antibody fragment expressed. Although it was unknown whether light chain-only or heavy chain-only dimers would be able to bind β-galactosidase, it was assumed that activity corresponded to the amount of properly folded, assembled heavy chain-light chain dimer. Soluble and cell free broth fractions from each of the 6 strains were tested for binding activity, as illustrated in FIG. 8. The antibody fragment expressed from pDOW1196 showed significantly higher activity than those expressed from pDOW3716 and pDOW3717. Over-expression of FrnE did not have an impact on the amount of activity detected in the soluble or cell free broth fractions of pDOW1196 containing strains. FrnE appears to have improved the amount of active Fab expressed from pDOW3716 and pDOW3717, with approximately two-fold higher activity detected.

### Example 5

### Expression of Fab Variants in DASGIP Mini-Bioreactors

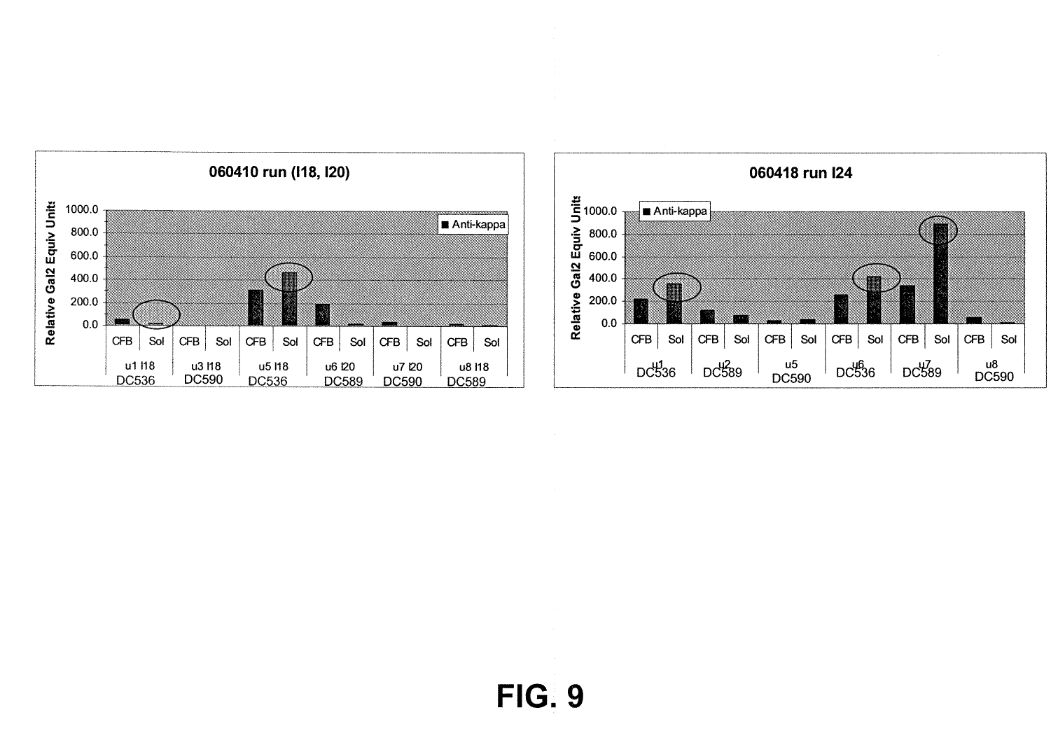

Fab expression from each of the three Fab constructs in the wild-type host DC454 was confirmed at the 300 mL fermentation scale in DASGIP mini-fermentors. As shown in FIG. 9, in three of four replicates, DC536 (carrying pDOW1196) expressed a significant amount of active Gal2 Fab in the cell free broth (CFB) and/or in the soluble fraction. Strain DC589 (pDOW3716) showed little if any active Fab expression in any of the four replicate experiments, whereas strain DC590 (pDOW3717) showed significant activity in one of four experiments performed. These results confirmed those observed at shake flask scale, with the highest amount of active Fab detected from DC536, which expresses the truncated Fab from pDOW1196.

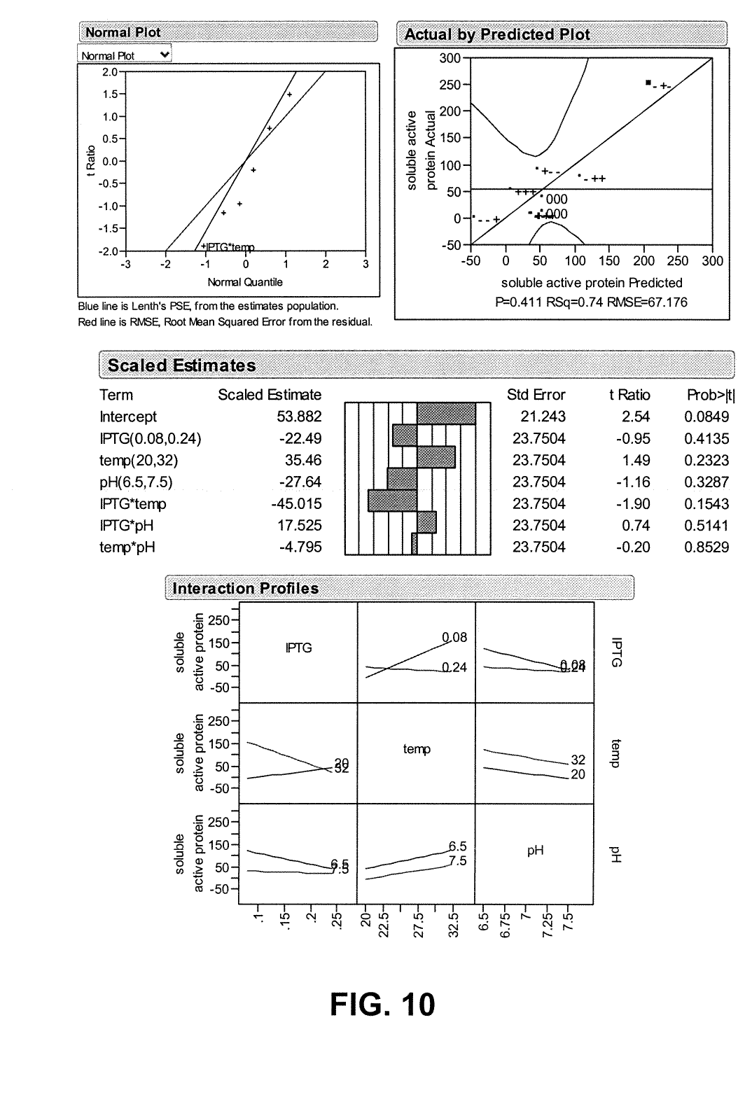

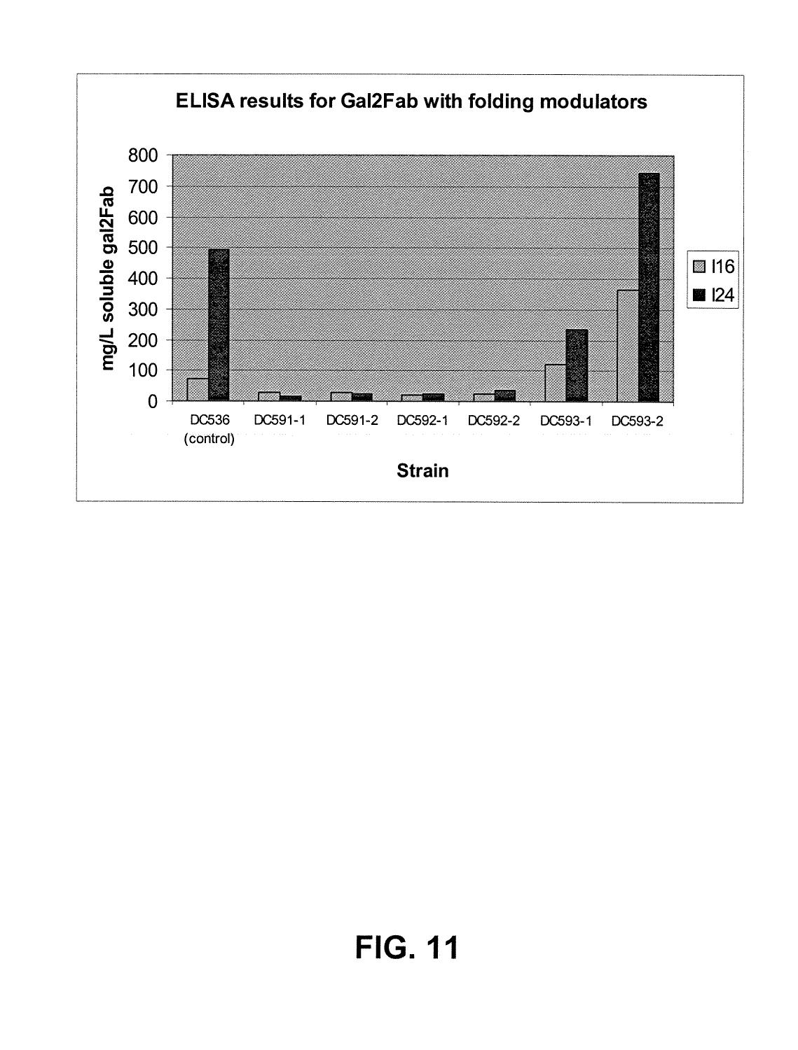

A design of experiment (DoE) analysis was performed using DC536 to determine whether fermentation conditions could be optimized to further improve the amount of soluble active Gal2 Fab expressed. The conditions tested are shown in Table 1. The analysis of the response (soluble active protein) indicated a normal distribution of data, as depicted in FIG. 10. The results all fell within the limits of confidence when plotted against the predicted results. The most significant effect on the expression of soluble active protein was seen in the interaction of IPTG concentration and induction temperature. Effects are visualized by the relationship of the lines in each box. Parallel lines indicate no effect interaction between the two graphed factors. Intersecting lines show that the two factors have a greater effect on response in combination. Interaction profiles indicate no effect interaction between the factors “induction pH” and “induction temperature” and very little effect interaction between “IPTG concentration” and “induction pH.” The interaction effect between “IPTG concentration” and “induction temperature” shows the highest significance.

Each of three strains over-expressing the frnE disulfide isomerase (DC591, DC592, DC593) was grown in duplicate in DASGIP mini-fermentors. Growth of these strains, when compared to the control (DC536), was comparable during the growth phase. During late induction phase the OD fell in the frnE over-expression strains, a possible indication of cell lysis. Final samples were viscous, causing difficulties in getting a discrete separation during sample collection and preparation. The viscosity of the samples may have contributed to the inconsistency in the ELISA results, as shown in FIG. 12, from duplicates of strain DC593 by allowing some protein to be sequestered in the CFB rather than the cell pellet. However, it was determined that this viscosity would not likely account for the significant difference in amount of protein between strains DC591/592 and strain DC593, as all frnE over-expression strains were viscous at harvest time. Strain DC593 expressed more Gal2 Fab than the control, DC536, in one of the two replicates. The second DC593 replicate expressed less Gal2 Fab than the control, but still much more protein than DC591 or DC592, as determined by ELISA (FIG. 12). Regardless of the variance in the DC593 replicates, it is clear that expression of the truncated Gal2 Fab from pDOW1196 resulted in increased yield of soluble active protein (DC536 and DC593) compared to expression of Gal2 Fab containing additional cysteine residues from pDOW3716 (DC589 and DC591) or from pDOW3717 (DC590 and DC592).

What is claimed is:

1. A method of improving expression of active antibody fragments comprising:
providing an antibody fragment (Fab);
truncating a heavy chain constant region (CH1) of a Fab to form a Fab fragment, wherein a cysteine amino acid required for disulfide bond formation with the light chain is removed;
cloning the Fab fragment in a prokaryote; and
expressing the Fab fragment in a prokaryote.

2. The method of claim 1, wherein the prokaryote comprises a bacteria.

3. The method of claim 3, wherein the prokaryote comprises a Pseudomonas strain.

4. The method of claim 3, wherein the prokaryote comprises Pseudomonas fluorescens.

5. The method of claim 1, wherein truncating the CH1 of a Fab comprises removing up to 100 amino acids upstream or downstream of the cysteine amino acid required for disulfide bond formation.

6. The method of claim 1, wherein truncating the CH1 of a Fab comprises removing up to five amino acids upstream or downstream of the cysteine amino acid required for disulfide bond formation.

7. The method of claim 1, wherein truncating the CH1 of a Fab comprises removing four amino acids upstream of the cysteine amino acid required for disulfide bond formation.

8. The method of claim 1, wherein the Fab fragment is cloned as a single operon transcribed from a plasmid promoter.

9. The method of claim 8, wherein the plasmid promoter is a Ptac promoter of plasmid pDOW 1169.

10. The method of claim 1, further comprising fusing a polymer, molecule or peptide to the Fab fragment.

11. The method of claim 10, wherein the molecule is selected from the group consisting of drugs, toxins, proteins, peptides, enzymes, polymers, nucleic acids, fragments, and derivatives thereof.

12. The method of claim 1, further comprising incorporating the Fab fragment into a pharmaceutical composition.

13. The method of claim 12, wherein the Fab fragment further comprises a peptide or molecule.

14. An expression vector comprising the following operably linked elements:
a transcription promoter;
a DNA segment encoding a Fab fragment having a truncated heavy chain constant region (CH1), wherein a cysteine amino acid required for disulfide bond formation with the light chain is removed; and
a transcription terminator.

15. The expression vector of claim 14, wherein the promoter is a Ptac promoter of plasmid pDOW 1169.

16. The expression vector of claim 14, further comprising:
a ribosome binding site after the promoter; and
a periplasmic secretion leader coding sequences fused to truncated heavy chain and light chain coding sequences.

17. A host cell comprising the expression vector of claim 14.

18. The host cell of claim 17, wherein the host cell comprises a microbe.

19. The host cell of claim 18, wherein the host cell comprises a Pseudomonas strain.

20. The host cell of claim 19, wherein the host cell comprises Pseudomonas fluorescens.

21. A method of improving expression of active antibody fragments comprising:
providing an antibody fragment (Fab);
truncating a kappa or lambda light chain of a Fab to form a Fab fragment;
cloning the Fab fragment in a prokaryote; and
expressing or secreting the Fab fragment in a prokaryote.

22. An expression vector comprising the following operably linked elements:
a transcription promoter;
a ribosome binding site after the promoter;
a DNA segment encoding a Fab fragment having a truncated kappa light chain or a truncated lambda light chain;
a periplasmic secretion leader coding sequences fused to truncated heavy chain and light chain coding sequences; and
a transcription terminator.
